(19) United States
(12) Patent Application Publication
(10) Pub. No.: US 2024/0407752 A1
(43) Pub. Date: Dec. 12, 2024

(54) SYSTEMS AND METHODS FOR MACHINE LEARNING BASED ULTRASOUND ANATOMY FEATURE EXTRACTION

(71) Applicants: THE TRUSTEES OF COLUMBIA UNIVERSITY IN THE CITY OF NEW YORK, New York, NY (US); WISCONSIN ALUMNI RESEARCH FOUNDATION, Masison, WI (US); TUFTS MEDICAL CENTER, Boston, MA (US)

(72) Inventors: Kristin M. Myers, New York, NY (US); Sachin Jambawalikar, New York, NY (US); Qi Yan, New York, NY (US); Alicia B. Dagle, New York, NY (US); Yucheng Liu, Fort Lee, NJ (US); Ronald Wapner, Medina, PA (US); Helen Feltovich, Madison, WI (US); Michael House, Boston, MA (US)

(73) Assignees: THE TRUSTEES OF COLUMBIA UNIVERSITY IN THE CITY OF NEW YORK, New York, NY (US); WISCONSIN ALUMNI RESEARCH FOUNDATION, Madison, WI (US); TUFTS MEDICAL CENTER, Boston, MA (US)

(21) Appl. No.: 18/738,863

(22) Filed: Jun. 10, 2024

Related U.S. Application Data
(60) Provisional application No. 63/507,397, filed on Jun. 9, 2023.

(51) Int. Cl. A61B 8/12; A61B 5/00

(52) U.S. Cl. CPC A61B 8/12; A61B 5/4343

(57) ABSTRACT

The disclosed subject matter provides systems and methods for predicting a spontaneous preterm birth based on transvaginal ultrasound images of a subject. An example method can include providing a preterm birth prediction model, obtaining one or more transvaginal ultrasound images of the subject, each including cervical features, determining measurements of a plurality of cervical structure features from the one or more ultrasound images, assessing, using the preterm birth prediction model, cervical health of the subject based on the measurements of the plurality of cervical structure features, and calculating the spontaneous preterm birth risk based on the assessed cervical health, using the preterm birth prediction model.

### CROSS-REFERENCE TO RELATED APPLICATION

This application claims priority to the U.S. Provisional Application Ser. No. 63/507,397, filed Jun. 9, 2023, the contents of which are hereby incorporated by reference in its entirety.

### GRANT INFORMATION

This invention was made with government support under 2036197 awarded by the National Science Foundation. The government has certain rights in the invention.

### BACKGROUND

Preterm birth (PTB), defined as delivery before 37 weeks of gestation, is the leading cause of perinatal death and a major contributor to long-term disabilities and elevated healthcare costs. With persistently high global rates of PTB and 15 million premature births yearly, PTB is a major public health problem with a high societal and financial burden. Most instances (80%) of preterm births are spontaneous (sPTB). Prediction can be difficult, especially among patients without a history of sPTB, hindering the development of interventions.

Ultrasound biomarkers explored for the prediction of sPTB includ cervical length (CL), anterior uterocervical angle (AUCA), lower uterine segment (LUS) thickness, and cervical funneling, with CL a reproducible, stand-alone predictor of sPTB. Accordingly, the clinical standard is transvaginal ultrasound cervical length (TVU CL) measured from B-mode images. While CL can an important predictor of sPTB, with a shorter CL conferring a higher risk of sPTB, the positive predictive value (PPV) of CL alone is limited, ranging from 26-52% for women with no history of sPTB.

Accordingly, there is an unmet need for improved detection of PTB.

### SUMMARY

The disclosed subject matter provides methods for predicting a spontaneous preterm birth based on transvaginal ultrasound images of a subject. An example method can include providing a preterm birth prediction model, obtaining one or more transvaginal ultrasound images of the subject, each including cervical features, determining measurements of a plurality of cervical structure features from the one or more ultrasound images, assessing, using the preterm birth prediction model, cervical health of the subject based on the measurements of the plurality of cervical structure features, and calculating, using the preterm birth prediction model, the spontaneous preterm birth risk based on the assessed cervical health.

In certain embodiments, the preterm birth prediction model can include a deep learning algorithm to identify the cervical shape, size and load information. In non-limiting embodiments, the deep-learning algorithm can be trained on a plurality of biomechanical records to extract shape features and compare extracted shape features against expert-reported features. The plurality of biomechanical records can include cervical features.

In certain embodiments, the one or more transvaginal ultrasound images can include unique pixel color values indicating segmentation of geometric features.

In certain embodiments, the determining measurements of a plurality of cervical structure features can include determining measurements of a cervical length, a lower uterine segment thickness, a cervical diameter, an anterior cervical diameter, a posterior cervical diameter, an anterior uterocervical angle, or combinations thereof. In non-limiting embodiments, the determining measurements of the cervical length further include determining a distance between an internal and an external end of a cervical canal of the subject.

In certain embodiments, the determining measurements of the anterior cervical diameter can further include a measurement along the cervical length. In non-limiting embodiments, the determining measurements of the posterior cervical diameter can further include a measurement along the cervical length. In non-limiting embodiments, the determining measurements of the cervical diameter can include a measurement at an intersection of the anterior cervical diameter and the posterior cervical diameter.

In certain embodiments, the determining measurements can include determining a perpendicular slope to the lower uterine segment and a midpoint of a posterior boundary of the subject's bladder. In non-limiting embodiments, the determining measurements of the lower uterine segment thickness can include a measurement between the midpoint of the bladder and an intersection between a perpendicular line to the lower uterine segment.

In certain embodiments, the determining measurements of the anterior uterocervical angle can include a measurement of an angle between the subject's anterior uterus and the cervix.

In certain embodiments, the method can further include measuring the cervical stiffness of the subject using a thin aspiration tube applied during a prenatal pelvic exam, and the assessing cervical health of the subject can be based on the measurements of the plurality of cervical structure features and the measured cervical stiffness.

In certain embodiments, the calculating the spontaneous preterm birth risk can include generating a risk score.

The disclosed subject matter provides systems for predicting a spontaneous preterm birth based on transvaginal ultrasound images of a subject. An example system can include a processor configured to provide a preterm birth prediction model based on a plurality of biomechanical records including cervical features, obtain one or more transvaginal ultrasound images of the subject, each including cervical features, determine measurements of a plurality of cervical structure features from the one or more ultrasound images, assess, using the preterm birth prediction model, cervical health of the subject based on the measurements of the plurality of cervical structure features, and calculate the spontaneous preterm birth risk based on the assessed cervical health, using the preterm birth prediction model.

In certain embodiments, the preterm birth prediction model comprises a deep-learning algorithm to identify cervical shape, size and load information, and the deep-learning algorithm can be trained on the plurality of biomechanical records to extract shape features and compare extracted shape features against expert-reported features.

In certain embodiments, the determining measurements of a plurality of cervical structure features can include determining measurements of a cervical length, a lower uterine segment thickness, a cervical diameter, an anterior cervical diameter, a posterior cervical diameter, an anterior uterocervical angle, or combinations thereof.

In certain embodiments, the determining measurements of the cervical length can further include determining a distance between an internal and an external end of a cervical canal of the subject. In non-limiting embodiments, the determining measurements can include determining a perpendicular slope to the lower uterine segment and a midpoint of a posterior boundary of the subject's bladder.

In certain embodiments, the processor can be configured to measure cervical stiffness of the subject using a thin aspiration tube applied during a prenatal pelvic exam, and the assessing cervical health of the subject can be based on the measurements of the plurality of cervical structure features and the measured cervical stiffness.

### BRIEF DESCRIPTION OF THE DRAWINGS

It is to be understood that both the foregoing general description and the following detailed description are exemplary and are intended to provide further explanation of the disclosed subject matter. The patent or application file contains at least one drawing executed in color. Copies of this patent or patent application publication with color drawing(s) will be provided by the Office upon request and payment of the necessary fee.

FIG. 1 illustrates an example method for assembling the EMR, imaging database of transvaginal ultrasounds, and extracting cervix features in accordance with the disclosed subject matter.

FIG. 2 is a functional a diagram showing an example method of incorporating EMR metadata and anatomical structural data with CNN-derived imaging features in a risk prediction network in accordance with the disclosed subject matter.

FIG. 3 is a cohort flow chart in accordance with the disclosed subject matter.

FIGS. 4A-4D provide examples of images submitted to Cervical Length Education and Review (CLEAR) in accordance with the disclosed subject matter.

FIGS. 5A-5E provide images showing a segmentation labeling scheme in accordance with the disclosed subject matter.

FIG. 6 illustrates a CL model workflow in accordance with the disclosed subject matter.

FIG. 7 provides a graph showing the model performance on CLEAR test data in accordance with the disclosed subject matter.

FIG. 8 provides a graph showing the model performance on out-of-distribution test data in accordance with the disclosed subject matter.

FIGS. 9A-9G provide images showing the ensemble model segmentation on CLEAR test data in accordance with the disclosed subject matter.

FIGS. 10A-10D provide images showing the ensemble model segmentation on out-of-distribution test CL data in accordance with the disclosed subject matter.

FIG. 11 provides images showing the algorithm-reported CL displayed on out-of-distribution test data in accordance with the disclosed subject matter.

FIG. 12A-12C provide images showing example CL outputs with high percent error in accordance with the disclosed subject matter.

FIG. 13 is a graph showing a Bland-Altman CL plot in accordance with the disclosed subject matter.

FIGS. 14A-14D provide images showing examples of model segmentation in accordance with the disclosed subject matter.

FIG. 15 is a graph showing the normal distribution of CL measurements in accordance with the disclosed subject matter.

FIG. 16 is a graph showing the percent error in the CL algorithm in accordance with the disclosed subject matter.

FIG. 17 is a graph showing the ensemble model performance on CLEAR validation data in accordance with the disclosed subject matter.

FIGS. 18A-18C provide the individual model comparisons on varied cervical geometry om accordance with the disclosed subject matter.

FIGS. 19A-19D provide a diagram showing the attention to UNet model segmentation on CLEAR validation data in accordance with the disclosed subject matter.

FIGS. 20A-20D provide diagrams showing the nn-UNet model segmentation on CLEAR in accordance with the disclosed subject matter.

FIGS. 21A-21D provide diagrams showing the SegResNet model segmentation on CLEAR validation data in accordance with the disclosed subject matter.

FIGS. 22A-22D provide diagrams showing the basic UNet model segmentation on CLEAR validation data in accordance with the disclosed subject matter.

FIGS. 23A-23D provide diagrams showing the 2-unit Residual UNet model segmentation on CLEAR validation data in accordance with the disclosed subject matter.

FIGS. 24A-24D provide diagrams showing the 4-unit Residual UNet model segmentation on CLEAR validation data in accordance with the disclosed subject matter.

FIGS. 25A-25D provide diagrams showing the transformer UNet model segmentation on CLEAR validation data in accordance with the disclosed subject matter.

FIG. 26 provides diagrams showing the out-of-distribution test set images excluded from analysis in accordance with the disclosed subject matter.

FIG. 27 provides images showing example cervical features in accordance with the disclosed subject matter.

FIGS. 28A-28D provide images showing example expert levels in accordance with the disclosed subject matter.

FIGS. 29A-29C provide images showing a three-part pre-processing of images in accordance with the disclosed subject matter.

FIGS. 30A-30C provide images showing example classified segmentations in accordance with the disclosed subject matter.

FIGS. 31A-31D provide images illustrating finding cervical length for a case 1 image in accordance with the disclosed subject matter.

FIGS. 32A-32D provide images illustrating finding cervical length for a case 2 image in accordance with the disclosed subject matter.

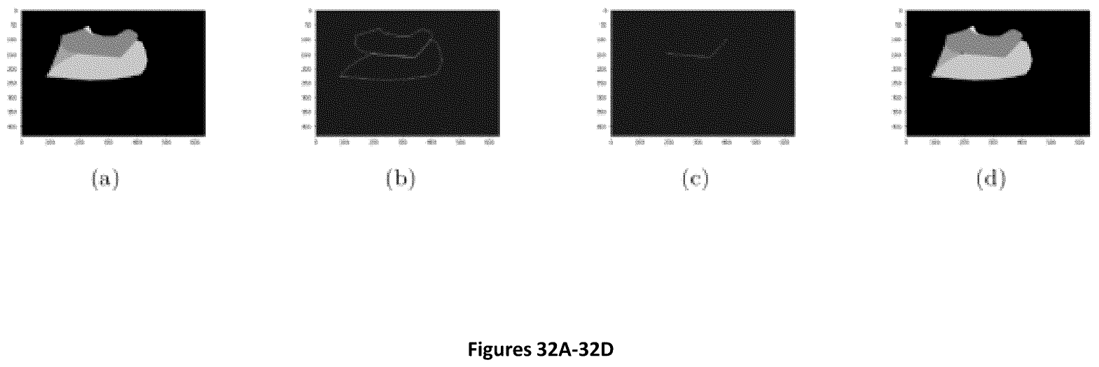

FIG. 33 provides images showing anterior and posterior cervical diameters measured at 25%, 50%, and 75% of cervical lengths in accordance with the disclosed subject matter.

FIGS. 34A-34C provide images illustrating the LUS thickness measurement in accordance with the disclosed subject matter.

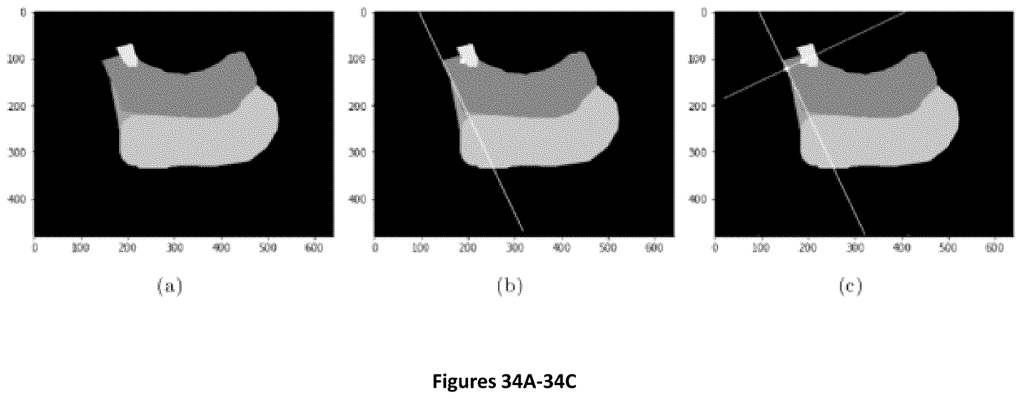

FIGS. 35A-35C provide images illustrating the AUCA thickness measurement in accordance with the disclosed subject matter.

FIGS. 36A-36C provide images showing the disclosed feature extraction tools in accordance with the disclosed subject matter.

FIG. 37 is a graph showing the comparison of clinical measurements and calculated measurements by the disclosed subject matter.

FIGS. 38A-38C illustrate finite element analysis (FEA) computer simulation that calculates the cervical tissue stress for various lower uterine thickness dimensions in accordance with the disclosed subject matter.

FIGS. 39A-39B provide diagrams showing FEA simulation calculating the cervical tissue stretch for various cervical elasticity values in accordance with the disclosed subject matter.

### DETAILED DESCRIPTION

The disclosed subject matter provides techniques for improved detection of preterm birth. The disclosed subject matter provides methods and systems for predicting a spontaneous preterm birth based on transvaginal ultrasound images of a subject. The disclosed subject matter can utilize the multi-class segmentation technique, distinguishing tissue regions enables algorithms to extract biomechanically relevant structural features of the maternal anatomy (e.g., LUS thickness, anterior/posterior cervical diameter, closed cervical area, CL and AUCA measurements).

Unless otherwise defined, all technical and scientific terms used herein have the same meaning as commonly understood by one of ordinary skill in the art. In case of conflict, the present document, including definitions, will control. Certain methods and materials are described below, although methods and materials similar or equivalent to those described herein can be used in the practice or testing of the presently disclosed subject matter. All publications, patent applications, patents, and other references mentioned herein are incorporated by reference in their entirety. The materials, methods, and examples disclosed herein are illustrative only and not intended to be limiting.

As used in the specification and the appended claims, the singular forms “a,” “an,” and “the” include plural referents unless the context clearly dictates otherwise. Thus, for example, a reference to “a compound” includes mixtures of compounds.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value.

An “individual” or “subject” herein is a vertebrate, such as a human or non-human animal, for example, a mammal. Mammals include, but are not limited to, humans, primates, farm animals, sport animals, rodents, and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

The disclosed subject matter provides methods for predicting a spontaneous preterm birth based on transvaginal ultrasound images of a subject. An example method can include providing a preterm birth prediction model, obtaining one or more transvaginal ultrasound images of the subject, each including cervical features, determining measurements of a plurality of cervical structure features from the one or more ultrasound images, assessing, using the preterm birth prediction model, cervical health of the subject based on the measurements of the plurality of cervical structure features, and calculating, using the preterm birth prediction model, the spontaneous preterm birth risk based on the assessed cervical health.

In certain embodiments, the biomechanical records can include a patient's electronic medical record. In non-limiting embodiments, the biomechanical records can include the cervical feature information of the subject and/or other patients. In non-limiting embodiments, the biomechanical records can be used to train the disclosed preterm birth prediction model.

In certain embodiments, the disclosed method can include determining measurements of a plurality of cervical structure features from the one or more ultrasound images. For example, one or more transvaginal ultrasound images of the subject can be obtained, and from the transvaginal ultrasound images, various cervical structure features (e.g., a cervical length, a lower uterine segment thickness, a cervical diameter, an anterior cervical diameter, a posterior cervical diameter, an anterior uterocervical angle, or combinations thereof) can be measured. The cervical structure features can be manually measured or by the disclosed preterm birth prediction model.

In certain embodiments, the cervical length can be measured by determining a distance between an internal and an external end of a cervical canal of the subject. In non-limiting embodiments, the anterior cervical diameter can be measured by a measurement along the cervical length. In non-limiting embodiments, the posterior cervical diameter can be measured by a measurement along the cervical length. In non-limiting embodiments, the cervical diameter can be measured by a measurement at an intersection of the anterior cervical diameter and the posterior cervical diameter.

In certain embodiments, a perpendicular slope to the lower uterine segment and a midpoint of a posterior boundary of the subject's bladder can be determined as a cervical structure feature. In non-limiting embodiments, the measurement of the lower uterine segment thickness can be determined by a measurement between the midpoint of the bladder and an intersection between a perpendicular line to the lower uterine segment.

In certain embodiments, the anterior uterocervical angle can be measured by measuring an angle between the subject's anterior uterus and the cervix.

In certain embodiments, the method can further include measuring cervical stiffness of the subject. For example, cervical stiffness can be measured using a thin aspiration tube applied during a prenatal pelvic exam, and the cervical health of the subject can be determined based on the measurements of the plurality of cervical structure features and the measured cervical stiffness. In addition to cervical shape and size, intrinsic cervical elasticity can also contribute to cervical structural integrity. The disclosed subject matter provides tools and techniques to measure cervical elasticity (i.e., stiffness). For example, the tools can include cervical aspiration and quantitative ultrasound. The aspiration tool can measure cervical tissue stiffness by applying a small negative pressure on the external cervical os and pulling the tissue (e.g., until it touches a 4 mm stop). The aspiration closure pressure can be recorded, where a higher value of closure pressure corresponds to a stiffer tissue.

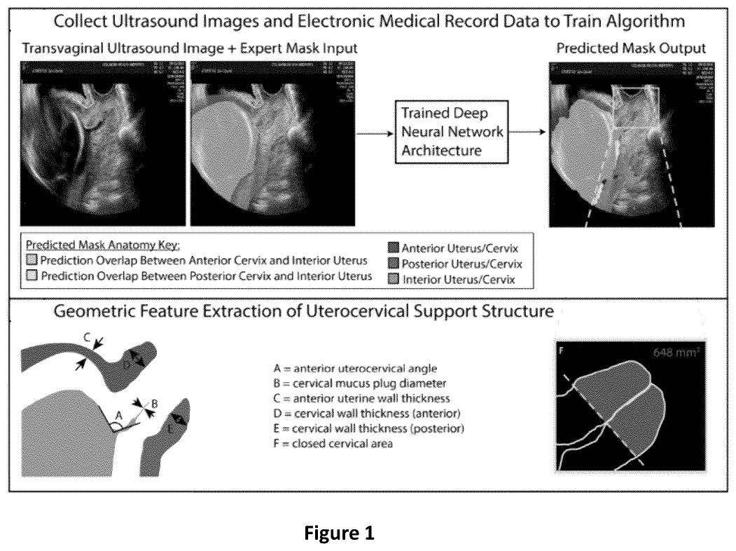

In certain embodiments, the preterm birth prediction model can be used to assess the cervical health of the subject based on the measurements of the plurality of cervical structure features and calculate the spontaneous preterm birth risk based on the assessed cervical health. In non-limiting embodiments, the preterm birth prediction model can include a deep learning algorithm to identify the cervical shape, size and load information. For example, the outcome of the disclosed model can be the anterior and posterior cervical segmented area, as depicted in the 2D transvaginal ultrasound plane (FIG. 1). This neural network predicted area (FIG. 1, upper right) can be compared to ground truth segmentations (FIG. 1, upper left), generated by expert sonographers, by using mean intersection over union (IoU) and DICE similarity coefficient. To validate this model compared to current standards of care, we can extract cervical length in addition to other measures (FIG. 1, bottom) from the disclosed model-generated mask and compare this to sonographer measurements taken from the raw ultrasound image.

In certain embodiments, statistical shape modeling can be used to further classify the shape of the cervix into different risk groups. In non-limiting embodiments, a dataset of patients can be created, containing clinical TVUS images, relevant patient electronic medical record (EMR) data and pregnancy outcomes.

In certain embodiments, the deep learning algorithm can be trained to automatically label cervix tissue and extract novel maternal shape features (e.g., cervical diameter, closed cervical area, lower uterine segment thickness) in addition to individual markers (e.g., cervical length and anterior uterocervical angle) from a routine clinical transvaginal ultrasound (TVUS) of pregnant patients (e.g., in 2nd trimester). In non-limiting embodiments, the disclosed neural network can be trained on CLEAR-certified transvaginal ultrasounds to trace the cervical geometry that can be unseen by TVUS images. Holistic cervical shape and lower uterine segment thickness can provide improved prediction of PTB than CL alone. In non-limiting embodiments, the disclosed neural network can be trained on TVUS images. Holistic cervical shape features and EMR data can provide improved predictions of PTB compared to CL alone.

In certain embodiments, TVUS segmentation, the disclosed subject matter can utilize multiple deep learning algorithms to generate pixel-level anatomical labels from transvaginal ultrasounds. For example, a multi-class residual UNet 2D CNN architecture can be used. The UNet 2D CNN architecture, a type of fully convolutional neural network (CNN) based on a contracting and expanding architecture with inter-unit (skip) connections, can be designed to integrate low-level positional features from the contracting path with high-level representations in the expanding path. This algorithm can be further optimized by adding residual connections for the ResUnet type of architecture, thereby stabilizing gradients during backpropagation. Other multiclass networks can be trained and optimized, including the DeepLabv3 CNN model and a combined UNet/Transformer-based architecture. This can allow for deeper networks with faster convergence and accuracy during multiclass or multilabel classification. In multiclass segmentation, the disclosed network can learn all segmentation classes and label the most likely class for each pixel, eliminating the overlapping regions (FIG. 1). The algorithm's accuracy can be evaluated using the DICE score metric between the automatic neural network generated and expert segmentations.

In certain embodiments, the disclosed deep-learning algorithm can be trained on the plurality of biomechanical records to extract shape features and compare extracted shape features against expert-reported features. For example, TVUS images can be used for training with expert segmented annotations of the 4 anatomical shapes. In non-limiting embodiments, TVUS images can include unique pixel color values indicating the segmentation of geometric features.

In certain embodiments, the disclosed subject matter can perform maternal anatomy feature extraction. For example, given the learned patient-specific mask output (FIG. 1, top), patient-specific anatomical features can be quantified (FIG. 1, bottom). In addition to cervical length, cross-cross sectional closed cervical area, utero-cervical angle, cervical mucus plug diameter, cervical wall thickness, and uterine wall thickness can be measured and utilized for the feature extraction. These programmable measurements of cervical size and shape, calculated from patient masks, can be employed as input features to the disclosed machine learning models as well as computational structural models of pregnancy.

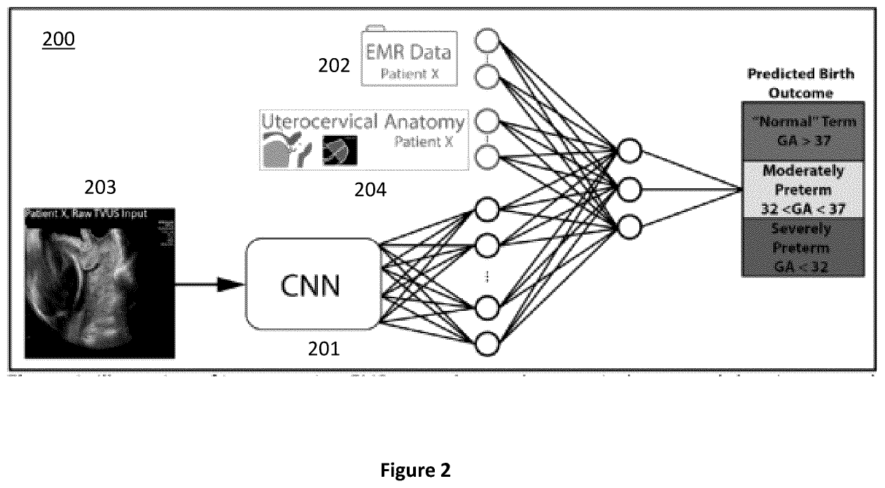

In certain embodiments, the disclosed subject matter provides a CNN-based feature extraction classifier 200. The CNN 201 based feature extraction classifier allows for combining contextual EMR features 202 (e.g., clinical history, demographic information, and laboratory data recorded in EMRs) with ultrasound imaging features 203 and the automatically derived anatomical features 204. This combined imaging, anatomical, and EMR feature network (FIG. 2) can be trained across collected patient data to predict higher and lower clinical risk groups. For example, a DenseNet architecture can provide 1024 features extracted from given images 203 to the fully connected layer for binary classification (0 or 1) of PTB or multi-class predictions (0=normal, 1=moderately preterm, 2=severely preterm). In non-limiting embodiments, non-imaging-based patient metadata 202 (EMR data: e.g., age, parity, BMI, etc.) and geometric features 204 can be introduced immediately before the fully connected layer as additional features to aid the classifier performance (FIG. 2).

In certain embodiments, specialized machine learning (e.g., data oversampling, weighted loss etc.) techniques can be performed for imbalanced data classification prediction to train and validate the disclosed algorithm.

In certain embodiments, after training of the disclosed network (FIG. 2), Local Interpretable Model-Agnostic Explanations (LIME) can be used to determine which variables have strong effects on PTB prediction. For example, in the disclosed model architecture, after processing the image through a CNN, imaging information can be summarized into a vector. Then, this vector can be concatenated with a vector of EMR and shape features. Therefore, the input that can be perturbed for the LIME analyses can be a vector of imaging information, EMR and shape features. If features from the vector of imaging information have a strong effect on PTB prediction, techniques such as integrated gradients and class activation maps can be utilized in the CNN substructure to provide a qualitative visual indication of which regions on the raw TVUS input image are most important in predicting preterm birth outcomes.

In certain embodiments, the method can include generating a risk score of the spontaneous preterm birth risk.

The disclosed subject matter also provides systems for predicting a spontaneous preterm birth based on transvaginal ultrasound images of a subject. An example system can include a processor that can be configured to perform the disclosed method. For example, the processor can be configured to provide a preterm birth prediction model based on a plurality of biomechanical records including cervical features, obtain one or more transvaginal ultrasound images of the subject, each including cervical features, determine measurements of a plurality of cervical structure features from the one or more ultrasound images. Assess, using the preterm birth prediction model, cervical health of the subject based on the measurements of the plurality of cervical structure features, and calculate, using the preterm birth prediction model, the spontaneous preterm birth risk based on the assessed cervical health.

In certain embodiments, the processor can be configured to operate the disclosed preterm birth prediction model. For example, the processor can include a deep learning algorithm to identify cervical shape, size and load information. The deep-learning algorithm can be trained on the plurality of biomechanical records to extract shape features and compare the extracted shape features against expert-reported features. In non-limiting embodiments, the disclosed model can include the CNN-based feature extraction classifier. The CNN-based feature extraction classifier can combine contextual EMR features (e.g., clinical history, demographic information, and laboratory data recorded in EMRs) with ultrasound imaging features and the automatically derived anatomical features. This combined imaging, anatomical, and EMR feature network can be trained across collected patient data to predict higher and lower clinical risk groups.

In certain embodiments, the processor can train the model by inputting a set of transvaginal ultrasound images and corresponding expert labels (e.g., manual segmentation of anatomy). During training, the disclosed model can be exposed to labeled images to identify patterns and features and iteratively learns by adjusting a set of variables, called hyperparameters. This fine-tuning of hyperparameters can inform the model performance until it achieves the best possible output (e.g., predicted segmentation of anatomy).

In certain embodiments, the processor can implement the model architecture (e.g., SegResNet, UNet, Residual UNet, nn-UNet, Attention UNet and Transformer UNet) using the Medical Open Network for Artificial Intelligence (MONAI) library, which can provide domain-specific capabilities for medical imaging.

In certain embodiments, the disclosed system can include the deep learning algorithm that can be trained on the plurality of biomechanical records to extract shape features and compare extracted shape features against expert-reported features.

In certain embodiments, the disclosed system can include the processor configured to determine measurements of cervical length, a lower uterine segment thickness, a cervical diameter, an anterior cervical diameter, a posterior cervical diameter, an anterior uterocervical angle, or combinations thereof. The disclosed processor can measure cervical length, a lower uterine segment thickness, a cervical diameter, an anterior cervical diameter, a posterior cervical diameter, an anterior uterocervical angle, or combinations thereof in accordance with the disclosed methods. For example, the cervical length can be measured by measuring the distance between an internal and an external end of a cervical canal of the subject. In non-limiting embodiments, the processor can be configured to determine a perpendicular slope to the lower uterine segment and a midpoint of a posterior boundary of the subject's bladder.

In certain embodiments, the processor can be configured to measure cervical stiffness of the subject using a thin aspiration tube applied during a prenatal pelvic exam. The assessing cervical health of the subject can be based on the measurements of the plurality of cervical structure features and the measured cervical stiffness.

In certain embodiments, the processor can be configured to generate a risk score of the spontaneous preterm birth risk using the disclosed PTB prediction model.

### Example 1: Deep Ensemble Multi-Class Segmentation of Cervical Anatomy and Automated Cervical Length Measurement on Transvaginal Ultrasound Images

The disclosed subject matter utilized multi-class segmentation methods, which can distinguish tissue regions enable algorithms to extract additional biomechanically relevant structural features of the maternal anatomy, including lower uterine segment (LUS) thickness, anterior/posterior cervical diameter and closed cervical area, as well as previously recorded cervical length (CL) and anterior uterocervical angle (AUCA) measurements. Measuring CL and labeling images with this level of detail is time-consuming, labor-intensive, and subject to inter-observer variation. An automated tool to label anatomy can allow more detailed and accurate extraction of geometric features. Patient variations in cervical geometry were evaluated during the second and third trimesters, and a novel tool was introduced to segment the entire 2D cervical region from TVU images into multiple anatomical classes, including anterior cervical tissue, posterior cervical tissue, and cervical canal space. This automated tool enables pixel-by-pixel predictions, and using a multi-class model helps identify boundaries between neighboring structures. It can serve as a teaching tool or a stand-alone resource in areas without access to experienced operators. Further, cervical shape mappings that identify and extract relevant features within the cervix and uterus can pinpoint key structural changes within these tissues during pregnancy to guide the investigation of the underlying molecular events, as well as enable creation of spontaneous preterm birth (sPTB) prediction models.

To build a standard AI-based model, the model was first trained by inputting a set of transvaginal ultrasound images and corresponding expert labels (manual segmentation of anatomy). During training, the model was exposed to labeled images to identify patterns and features and iteratively learned by adjusting a set of variables called hyperparameters. This fine-tuning of hyperparameters informs model performance until it achieves the best possible output (predicted segmentation of anatomy).

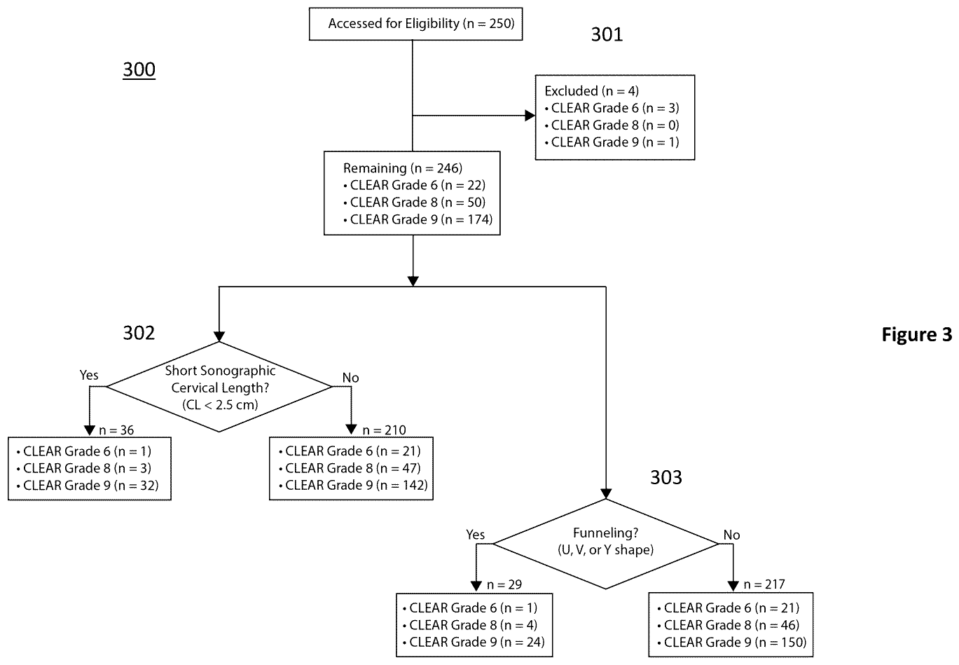

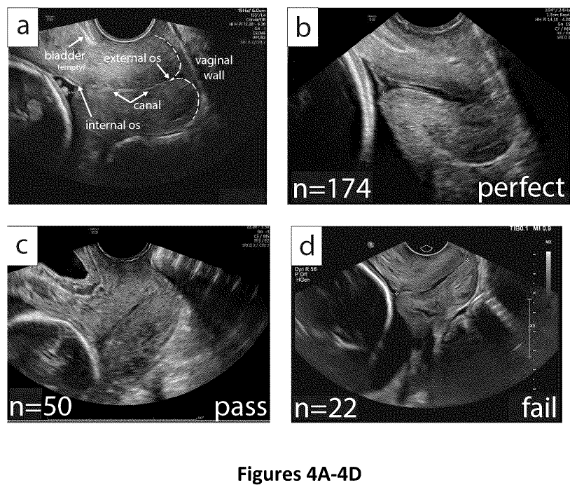

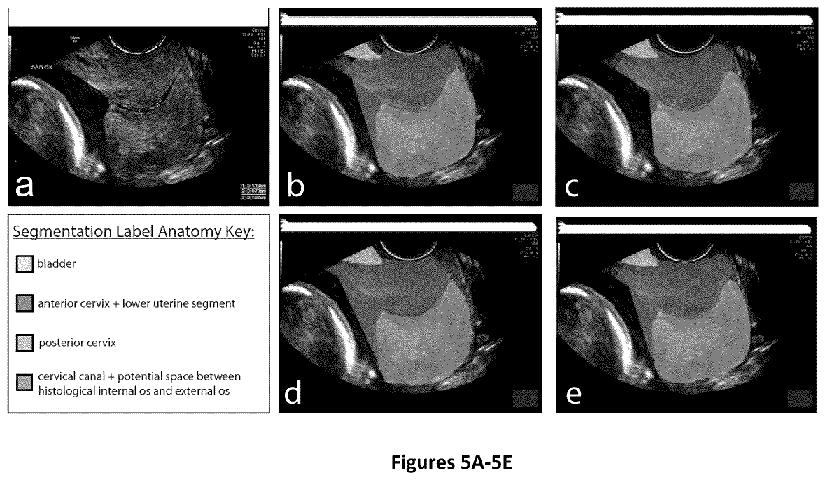

A diverse dataset (training, validation and reserved test set) of 250 deidentified TVU images submitted to the Cervical Length Education and Review (CLEAR) program was used (FIG. 3), collected from various centers and imaging systems across the USA. In FIG. 4, the following CLEAR criteria was included: 1) exam is transvaginal; 2) maternal bladder is empty; 3) field of view is optimized; 4) anterior cervical width=posterior cervical width; 5) internal os is well seen; 6) external os is well seen; 7) endocervical canal is visible in its entirety; 8) calipers are correctly placed; 9) shortest, best of 3 measurements is reported. In FIG. 3, the chart 300 illustrates the quantity of excluded data for model training and testing. The population is further categorized 302 based on short cervical length (<2.5 cm) and the presence of cervical funneling 303 (inclusive of grade 6, 8 and 9 images). Images were graded by CLEAR expert readers per defined criteria (FIG. 4, subpopulation details in FIG. 301). FIG. 4A shows the CLEAR-certified image with demarcations of anatomical landmarks. FIG. 4B shows the perfect grade 9/9 image satisfies all CLEAR criteria. FIG. 4C shows the passing grade 8/9 image does not satisfy criteria #2. FIG. 4D shows the failing grade 6/9 image does not satisfy criteria #3, 4, and 9. Training labels for the CLEAR dataset were generated via a majority choice voting system (FIG. 5). In FIG. 5, an unlabeled TVU image (5A) is labeled by 3 experts: 1 sonographer (5B) and 2 clinicians (5C, 5D) according to the segmentation key. The GT label, determined by a majority voting method applied to the 3 expert masks, is displayed (5E).

To further validate model performance and generalizability to a low-risk population, the algorithm was applied to an out-of-distribution test dataset of 30 pregnant patients at Intermountain Health (IH, Provo, UT). Images were collected at 22-25 weeks. One subject was removed from analysis due to sPTB, leaving 9 (31%) nulliparous and 20 (69%) multiparous. Of these, n=1 cervix was short (CL<2.5 cm). The out-of-distribution images were graded by only 1 of the previous experts, justified by high inter-rater agreement in the training dataset.

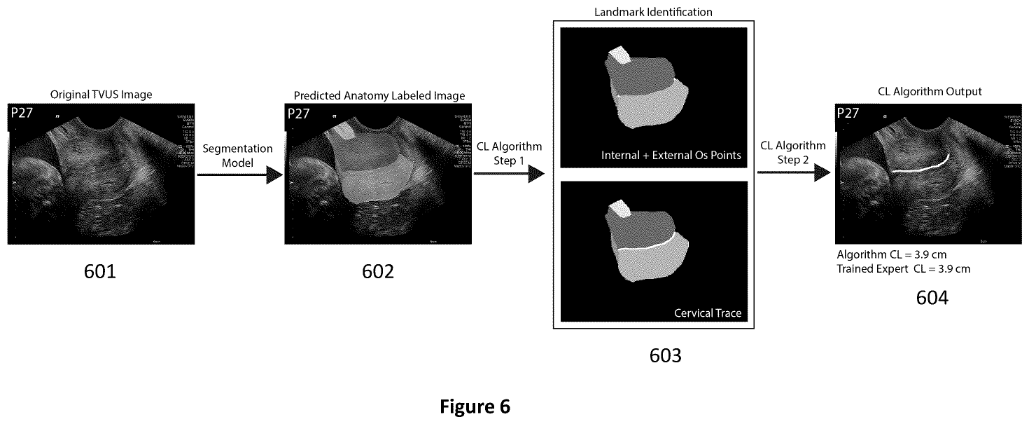

Data preprocessing and model training were performed in accordance with previous methods¹. The Medical Open Network for Artificial Intelligence (MONAI) library, which provides domain-specific capabilities for medical imaging, was used to implement the following segmentation model architectures: SegResNet, UNet, Residual UNet, nn-UNet, Attention UNet and Transformer UNet (model implementation detailed in Appendix B). Custom Python scripts were developed to automatically measure CL from segmentation masks (FIG. 6). In FIG. 6, the original, raw TVU image input (column 1) 601 is fed to the segmentation model to generate a predicted mask (column 2) 602 of the labeled anatomy, which is then provided as input to the CL extraction algorithm. During an intermediate procedure, the internal and external os are identified from the segmentation mask (column 3) in order to identify the cervical trace (column 3) 603 and then visualize this cervical trace feature overlaid on the original US image (column 4) 604 to measure CL.

Cervical Length Methodology: If the cervical canal class is present, the algorithm starts by finding internal os with the following method: 1) The algorithm locates the superior most boundary of the cervical canal+potential space class (shown in green); 2) These superior (or leftmost) points are fit to a line, and the image is rotated such that this line is oriented vertically; 3) The algorithm then counts the number of green points per column and calculates the derivative, which indicates how quickly the width of the cervical canal+potential space class changes; 4) The derivative is graphed lengthwise across the image and the first point where the derivative plateaus below a preset threshold are taken as the internal os location. Alternatively, if the cervical canal+potential space class is not present in the prediction image, the internal os location is derived from the leftmost point with adjacent anterior and posterior cervical tissue. The external os is then identified as the rightmost point of adjacent anterior and posterior cervical tissue. The cervical trace is finally taken as the adjacent anterior and posterior tissue between the internal and external os (FIG. 6). If a mucus plug is visible in the image and is labeled as the cervical canal class, the vertical midpoint of each column is taken as the point along the cervical trace.

Segmentation Model Hyperparameter Training (on CLEAR Dataset): The model computed a 5-channel output corresponding to the background and the 4 classes depicted in FIG. 5. The multiclass SegResNet and Transformer UNet (UNETR in MONAI) models were trained with varied dropouts, maintaining all other default parameters. Both the multi-class Residual UNet and multi-class Attention UNet architecture were trained with 5 convolutional layers (corresponding to 16, 32, 64, 128, and 256 channels) and a stride length of 2. The multi-class nn-UNet (DynUNet in MONAI) architecture was trained with a kernel size of 3 and a stride length of 2. During hyperparameter optimization, the number of residual units was varied only for the multiclass Residual UNet architecture. For each model architecture, Adam and SGD optimizers were considered. The learning rate varied from 0.001-0.01 for each optimizer, as shown in Table 1. Dropout of 0.1-0.4 was introduced for each model to decrease over-fitting. The SegResNet model was optimized with an Adam optimizer, learning rate=0.001, and dropout=0.3. The UNet model was optimized with an Adam optimizer, learning rate=0.05, and dropout=0.05. The Residual UNet architecture was optimized with an Adam optimizer, learning rate=0.001, and dropout=0.3. The nn-UNet model was optimized with an Adam optimizer, learning rate=0.001, and dropout=0.4. The Attention UNet (attn-UNet) model was optimized with an Adam optimizer, learning rate=0.001 and dropout=0.1. The Transformer UNet (UNETR) was optimized with an SGD optimizer, learning rate=0.1, and dropout=0.4.

During model training, Dice loss and Dice metric were monitored. An average Dice metric value was calculated for each epoch by averaging class-specific dice metrics across every class except the background. The model was allowed to run for 50 epochs during training, and early stopping was applied to monitor the validation loss with a patient of 5 epochs. The model checkpoint with the best average Dice metric on the validation set during training was saved. Predictions were generated by feeding inputs through the trained model, applying SoftMax activation along the class dimension and reporting the arg max value along the class dimension to determine the predicted class of each pixel in an image.

All models were run on a single Tesla V100-32 GB GPU. Model training was performed in Python 3.9, using PyTorch and MONAI packages.

CLEAR Dataset: Training identified the best-performing models for each architecture, iteratively checking performance on the validation dataset after each training procedure. Hyperparameters are indicated in Table 1. The depicted hyperparameter space was explored during model training on the CLEAR dataset. The experimentally derived best hyperparameters for each model type are indicated with a preceding asterisk (*item).

TABLE 1
Hyperparameters explored during model training.

| | Hyperparameters | | | |
|---|---|---|---|---|
| | Residual | | | |
| Model Type | Units | Optimizer | Dropout | Learning Rate |
| SegResNet | NA | *Adam, SGD | 0.1, 0.2, | 0.1, 0.05, |
| | | | *0.3, 0.4 | 0.01, *0.001 |
| UNet | NA | *Adam, SGD | 0.1, 0.2, | 0.1, *0.05, |
| | | | *0.3, 0.4 | 0.01, 0.001 |
| Residual | 2, *4 | *Adam, SGD | 0.1, 0.2, | 0.1, 0.05, |
| UNet | | | *0.3, 0.4 | 0.01, *0.001 |
| nnUNet | NA | *Adam, SGD | 0.1, 0.2, | 0.1, 0.05, |
| | | | 0.3, *0.4 | 0.01, *0.001 |
| attn-UNet | NA | *Adam, SGD | *0.1, 0.2, | 0.1, 0.05, |
| | | | 0.3, 0.4 | 0.01, *0.001 |
| UNETR | NA | Adam, *SGD | 0.1, 0.2, | *0.1, 0.05, |
| | | | 0.3, *0.4 | 0.01, 0.001 |

The predicted labels for these models were evaluated against ground truth using 3 similarity measures (Table 4), which assess how similar one image is to another image by comparing the pixel overlap (Dice Metric, Jaccard Index) or the degree of mismatch by assessing how far away one image representation is from another (Hausdorff distance). All 3 measures indicated that SegResNet, Residual UNet, Attention UNet, and nn-UNet were the highest-performing models. UNet and transformer UNet also offered strong model performance but had lower segmentation overlap scores. The Transformer UNet performed reasonably well, but the boundaries suffered from a pixelation-like quality. Statistical tests confirmed that each top-performing model (SegResNet, Residual UNet, Attention UNet and nn-UNet) differed with statistical significance (adjusted p<0.01) from the less well-performing models (UNet and transformer UNet).

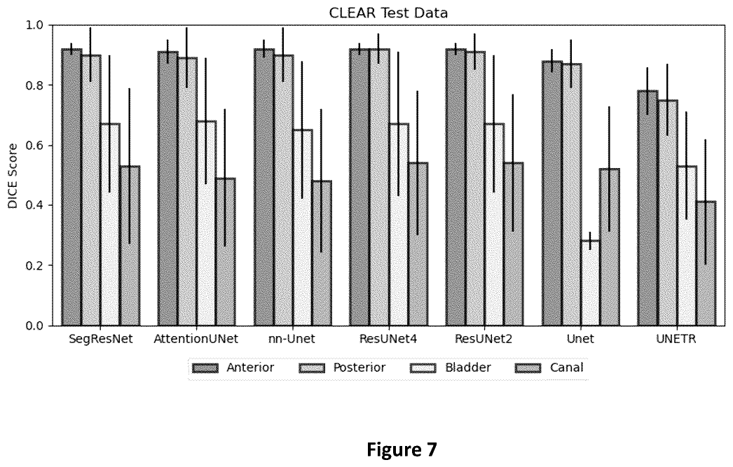

Since the 3 similarity measures serve the same purpose, the Dice Metric was used to further compare model performance on the reserved (CLEAR) test set simply because it is the most widely used. The dice score was plotted for each class across all model types (FIG. 7). In FIG. 7, all models are compared using class-specific Dice scores, averaged across all images in the reserved test set from the original CLEAR dataset. Error bars indicate 1 standard deviation from the mean across images in the test set. Models are ordered from left to right in terms of descending time required for model training.

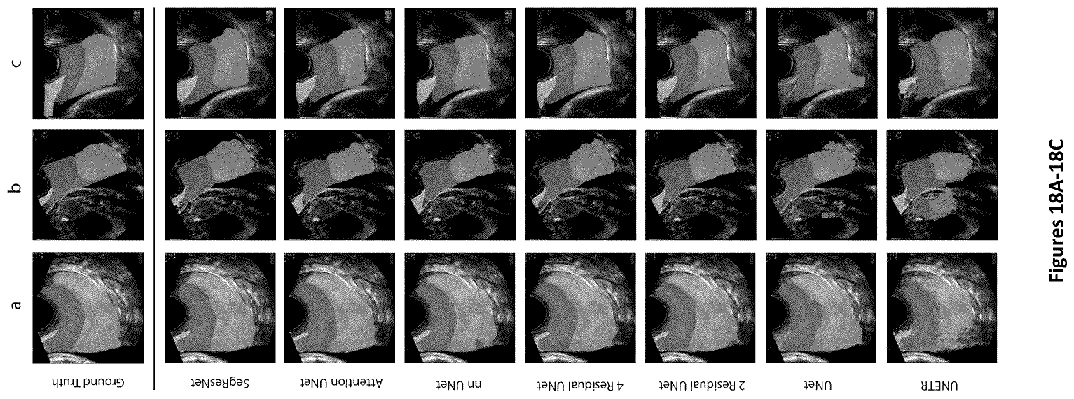

These models were plotted in descending order from left to right with respect to the time required for training (FIG. 18). The 4 best-performing architectures (SegResNet, Attention UNet, nn-UNet, and Residual UNet) also had the lowest training time (Table 5), indicating the sufficiency of these fewer complex models.

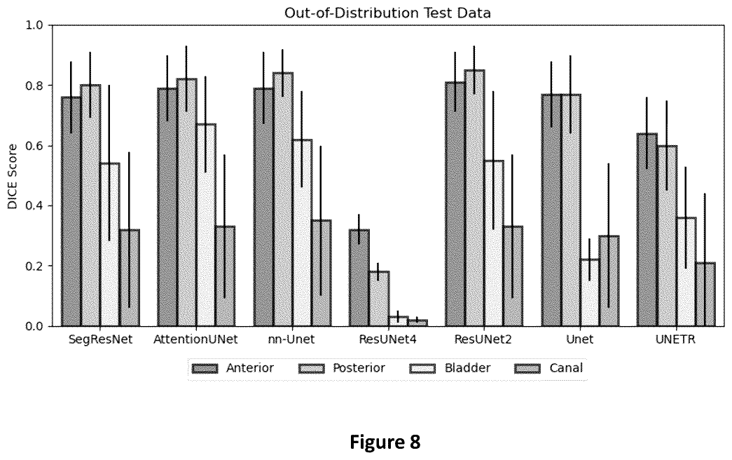

Out-of-distribution Dataset: To interrogate generalizability, models were evaluated on the separate out-of-distribution cohort from IH, comparing performance using class-specific Dice scores (FIG. 8). In FIG. 8, all models are compared using average Dice scores across individual classes, with bars showing standard deviation across images in the out-of-distribution test set (N=29, Utah USA). Models are ordered from left to right in terms of descending time required for model training. As expected with the application of a model to an out-of-distribution test dataset, all models experienced a small performance drop compared to the CLEAR reserved test set. The 4-unit Residual UNet likely overfit the CLEAR dataset, evidenced by the large drop in segmentation performance. Since this demonstrates a lack of generalizability, it was excluded from further analysis. The 4 best performing models maintained high Dice scores of approximately 0.8 for anterior and posterior cervix classes. Of these, the 2-unit Residual UNet had the highest model performance on the out-of-distribution test dataset (Dice scores: 0.81 and 0.85 on the anterior and posterior cervix, respectively), the nn-UNet (0.79 and 0.84) and attention UNet (0.79 and 0.82) performed similarly well and the SegResNet (0.76 and 0.80) performed slightly less well.

Final Model Selection: Among the 4 best performing individual models, no single model outperformed the others on the reserved or out-of-distribution test sets. Therefore, an ensemble approach was used to leverage the strength of each model and mitigate pixel-wise segmentation errors of individual predictions, thereby improving overall performance and reducing the risk of over-fitting to the training dataset. This method concatenates all 4 best-performing model outputs and employs pixel-wise voting to determine the final model output. Per majority voting, our ensemble model incorporated 3 out of the 4 best performing models. This demonstrated improvement in Dice score as compared to individual models (Tables 2 and 4).

TABLE 2
Comparisons of ensemble model performance.

| | Class | | | | |
|---|---|---|---|---|---|
| | | | Anterior | Posterior | Cervical |
| Model | Background | Bladder | Cervix | Cervix | Canal |
| aUNet, nnUNet, | 0.98 ∓ | 0.66 ∓ | 0.92 ∓ | 0.91 ∓ | 0.55 ∓ |
| ResUNet | 0.02 | 0.23 | 0.03 | 0.08 | 0.24 |
| aUNet, nnUNet, | 0.98 ∓ | 0.67 ∓ | 0.93 ∓ | 0.91 ∓ | 0.55 ∓ |
| SegResNet | 0.02 | 0.22 | 0.02 | 0.09 | 0.23 |
| aUNet, | 0.98 ∓ | 0.68 ∓ | 0.92 ∓ | 0.91 ∓ | 0.56 ∓ |
| SegResNet, | 0.02 | 0.21 | 0.02 | 0.08 | 0.24 |
| ResUNet | | | | | |
| nnUNet, | 0.98 ∓ | 0.66 ∓ | 0.92 ∓ | 0.91 ∓ | 0.57 ∓ |
| SegResNet, | 0.02 | 0.22 | 0.02 | 0.08 | 0.24 |
| ResUNet | | | | | |

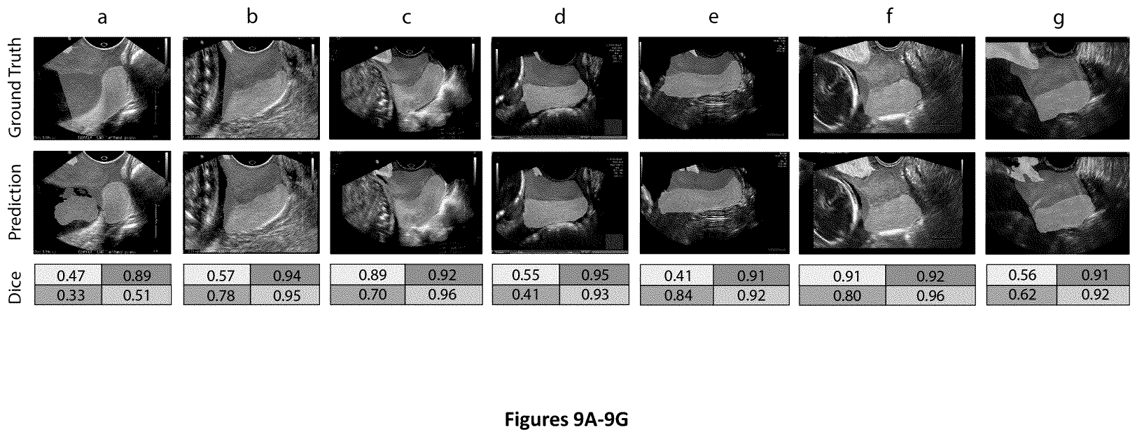

The combination of attention UNet, nn-UNet and SegResNet was used for the final evaluation. Similar model performance was observed across all 4 combinations of 3 models, but this combination achieved a higher anterior cervix Dice score on the reserved test set. This ensemble model was thus used to generate predictions for the reserved and out-of-distribution test sets, both of which demonstrated that the model generalizes well to new data. When applied to the reserved test set, the model performed well across diverse cervical etiologies, such as cervices that were of average length/width, curved, linear, long, short/squat, funneled, and adjacent to a full bladder (FIG. 9). Across all reserved test set images, the model achieved a high anterior and posterior cervix Dice score of roughly 0.93 and 0.91, respectively. In FIG. 9, within the original reserved test set, ground truth and predictions from the combined model are shown, calculated by a majority vote of attention UNet, nn-Unet and SegResNet. The model segmented cervical tissue well across diverse cervical etiologies, including: (9A) a large cervical funnel, (9B) an average length/width cervix, (9C) a curved cervix, (9D) a linear cervix, (9E) a long cervix, (9F) a short/squat cervix and (9G) a cervix with an adjacent full bladder.

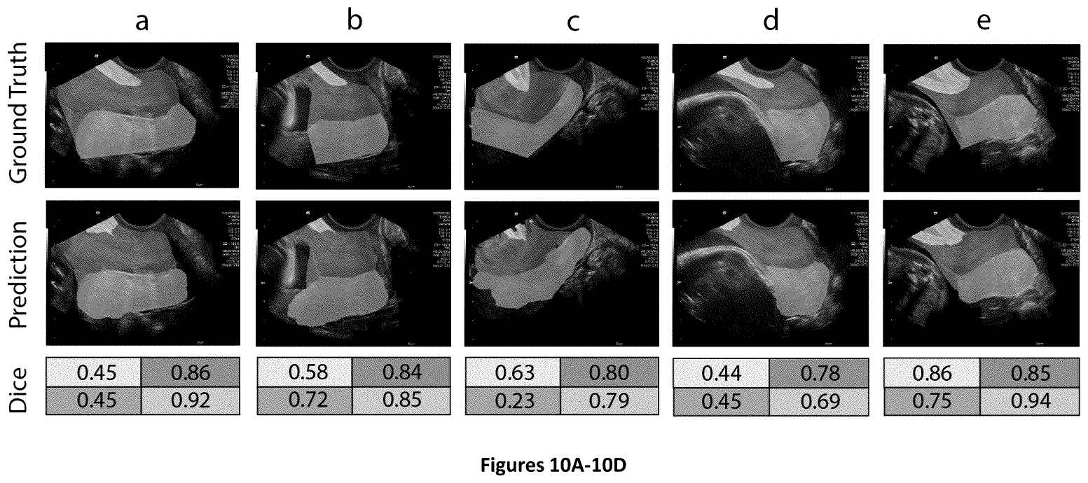

Evaluation of the out-of-distribution dataset indicated similarly high model performance for the aforementioned diverse cervical etiologies as well as in the presence of fetal anatomy near the internal os (FIG. 10). On this out-of-distribution dataset, the Dice score dropped slightly to 0.80 and 0.84 for the anterior and posterior cervix classes, respectively; however, visual inspection of the prediction images confirmed high model performance. In FIG. 10, within the out-of-distribution test set, ground truth and predictions from the combined model are shown, calculated by a majority vote of attention UNet, nn-Unet and SegResNet. When evaluated on this previously unseen dataset, the model performed well across diverse cervical etiologies, including: (10A) an average length/width cervix, (10B) a short/squat cervix, (10C) a long/curved cervix, (10D) fetal anatomy placed near the internal os of the cervix, and (10E) a full bladder pressing on the anterior cervix and lower uterine segment.

Interoperator Metrics: To evaluate inter-operator variability, measures of similarity were calculated between the majority ground truth label and each expert label on the test set. These metrics were then averaged across all experts to derive inter-operator values (Table 3). For the reserved test set, the inter-operator Dice score averaged across all classes except background was 0.82, with class-specific Dice scores of 0.94 for both anterior and posterior cervix classes. When evaluated on the reserved (CLEAR) test set, the combined model architecture achieved a high Dice score of 0.77 averaged across every class except the background, with class-specific Dice scores of 0.93 and 0.91 for the anterior and posterior cervix class, respectively, confirming that the model performed only slightly below the clinical expert agreement.

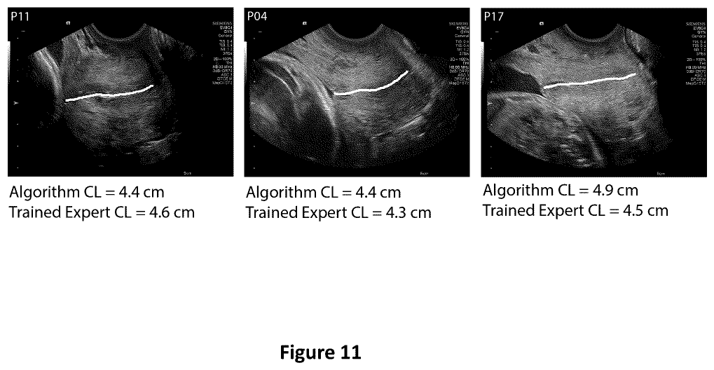

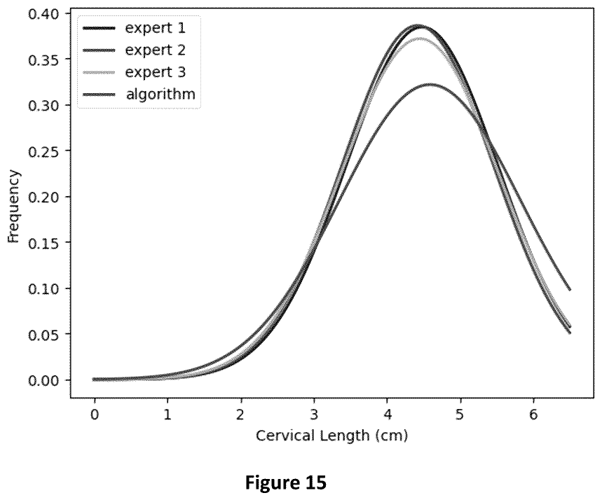

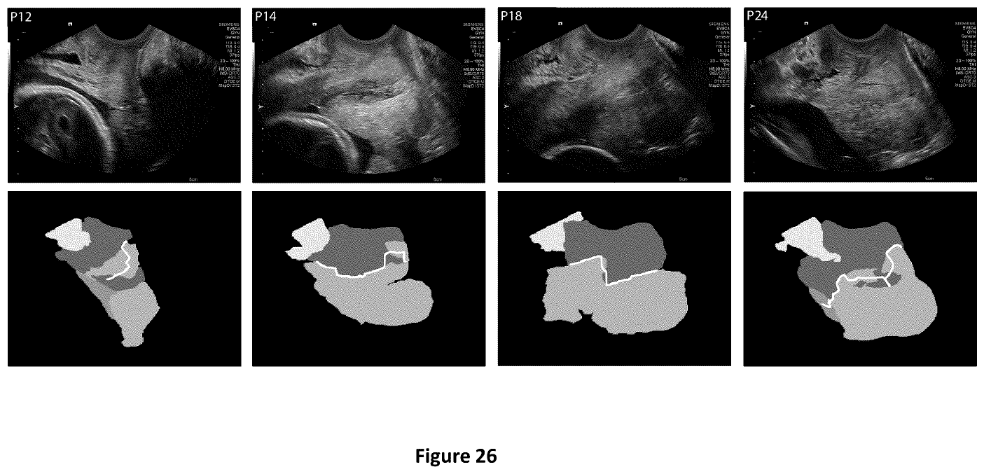

Cervical Length: The disclosed models accurately reproduce TVU CL (FIG. 11) with methods that leverage underlying geometry from the image inputs and predicted segmentation masks. In FIG. 11, the white line represents the algorithm's CL measurement overlaid on the TVU image. These examples showed nearly perfect agreement between the algorithm and the experts. Of the 29 patients in the out-of-distribution test dataset, 4 had anatomically improbable predicted segmentation labels (due to poor image quality) and were excluded from subsequent analysis (FIG. 26). For the remaining 25, CL was binned in 0.5 cm increments, and normal distributions were fit to histograms plotted for the algorithm and each sonographer (FIG. 15). Visually, statistical tests confirmed that reported values from the algorithm and experts are likely drawn from the same distribution, meaning they agree.

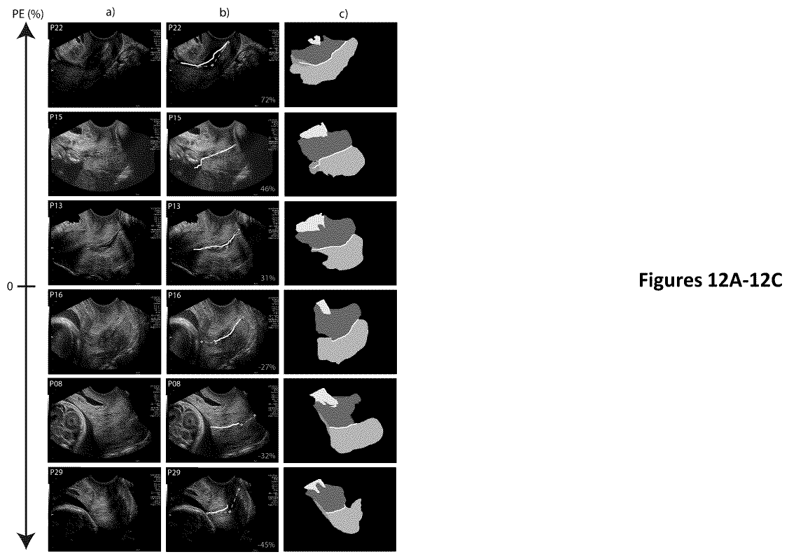

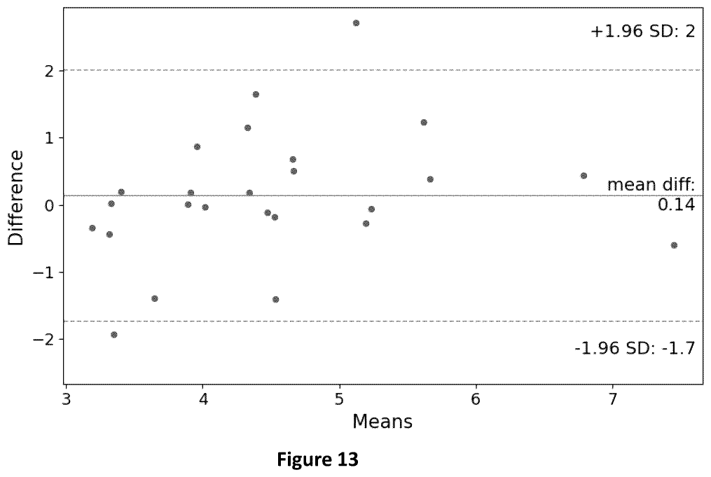

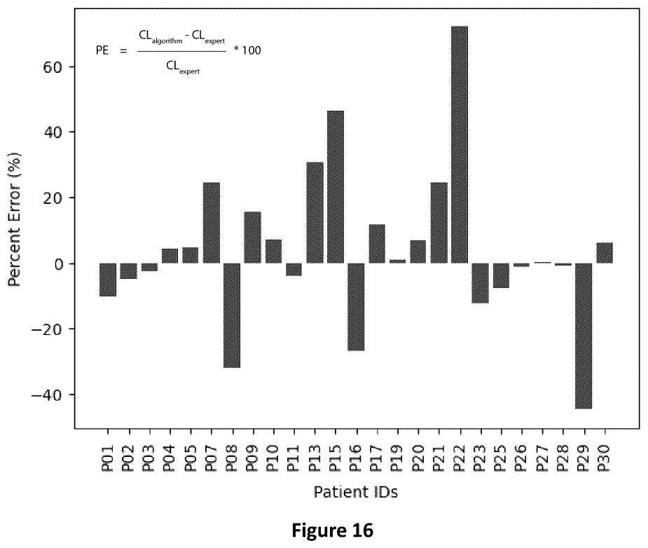

To further examine differences between algorithm and sonographer-reported values, the percent error was plotted for each patient across the dataset (FIG. 16). Examples with relatively high absolute error (PE<−25% or PE>25%) demonstrate CL measurements follow the underlying segmentation schemas and are susceptible to shadowing artifacts (FIG. 12). Bland-Altman plots comparing the CL measures from the algorithm against the expert measures (FIG. 13) indicate that expert readers and the algorithm can be used interchangeably to measure TVU CL, with a mean bias of 0.14 cm. In FIG. 12, all images with an absolute error greater than 25% are displayed in descending order from highest positive PE to lowest negative PE, including (12A) the original TVU image, (12B) the cervical prediction overlay in white with expert caliper label in green, and (12C) the cervical prediction overlay in white atop the predicted segmentation mask. In some cases (P08, P29), the presence of shadowing lends the algorithm to underpredict the cervical area, and this error propagates to cervical traces. Contraction of the lower uterine segment (P22) also leads to overprediction of cervical tissue and, therefore, errors in cervical length. The presence of a fetal foot is mistaken as cervical tissue (P15), which also leads to an incorrect internal os location, inflating the predicted cervical length. All cervical traces follow the trajectory of the cervical canal but are sometimes over- or under-predicted according to errors in the segmentation (column c). In FIG. 13, the plot compares the CL reported from the algorithm to the average value of the 3 expert measures for the out-of-distribution dataset. The mean bias of 0.14 suggests the automatic method slightly overestimates CL compared to the expert. The differences fall within a narrow range, indicating good agreement overall, with no significant outliers, which affirms the automatic CL method's consistency. The interoperator metrics for all 3 similarity metrics are reported here in Table 3. Inter-operator metrics were calculated by averaging Dice metric (DM), Hausdorff distance (HD), and Jaccard index (JI) across all 3 experts on reserved (CLEAR) test set images. Individual class values are reported in addition to the average across segmentation classes. In cases where Hausdorff distance was an infinite value for 1 expert, * indicates the average was calculated using the remaining 2 expert values.

TABLE 3
Inter-operator metrics.

| Inter- | Class | | | | | |
|---|---|---|---|---|---|---|
| operator | | | Anterior | Posterior | Cervical | |
| Metric | Background | Bladder | Cervix | Cervix | Canal | Average |
| DM | 0.98 | 0.82 | 0.94 | 0.94 | 0.59 | 0.85 |
| HD | 17.47 | *7.24 | 13.47 | 13.61 | *57.57 | *23.18 |
| JI | 0.97 | 0.74 | 0.88 | 0.89 | 0.47 | 0.79 |

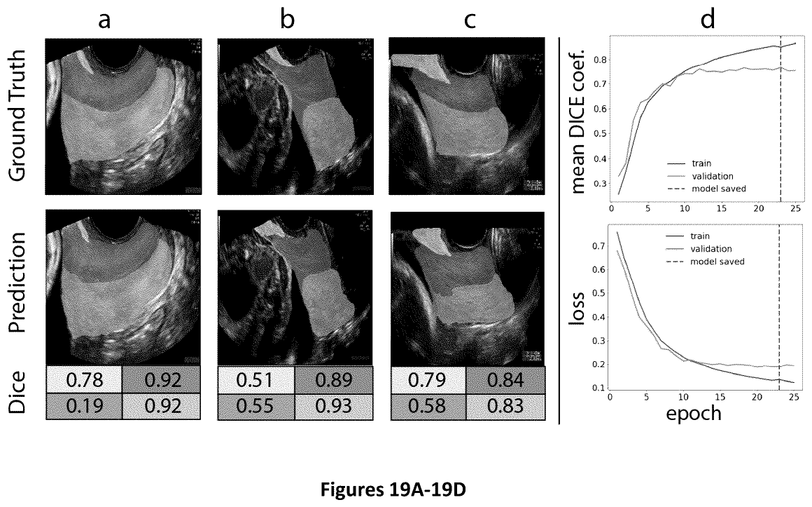

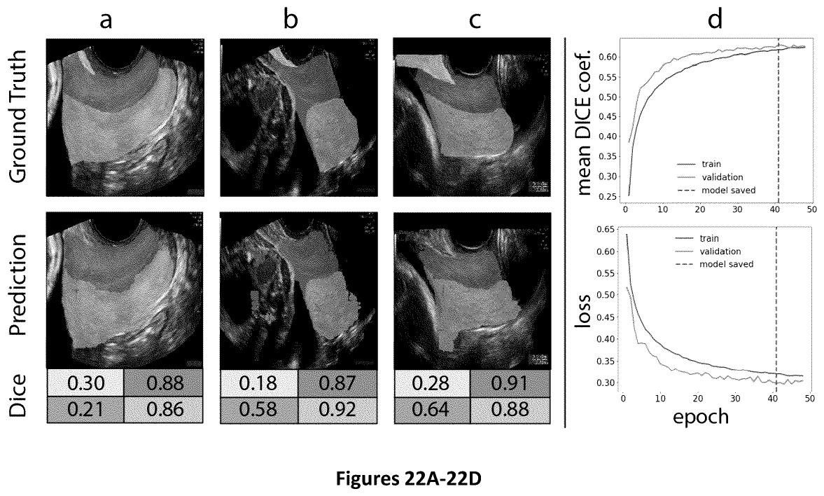

Segmentation Model: A detailed comparison of model performance, evaluated using the Dice score, can be found in Table 4 and FIG. 18, with model training times specified in Table 5. The 4 best performing models (Attention UNet, nn-UNet, SegResNet and UNet with 2 residual units) are further described in Tables 6-9, reporting multiple similarity metrics: Dice Score, Jaccard Index and Hausdorff distance. Diverse cervical etiologies (long/curved, short/squat and median length/width) are then used as a visual, qualitative comparison between model performance; FIGS. 19-26 display model predictions overlaid on each of these 3 services for each of the 6 models depicted in Table 4. Further inspection of test images indicated that the basic UNet with no residual units under-predicts the bladder class, as shown in FIG. 22.

Because individual models had similar performance, an ensemble model approach was used, which is explained in more detail here. Specifically, the 3 pre-trained models generate a segmentation mask and agreement is evaluated on an individual pixel-level basis—if at least 2 models agree on a pixel assignment, the consensus value is assigned as the final pixel label, similar to the methodology used in ground truth data creation.

TABLE 4
Comparisons of individual model performance; model comparisons based
upon Dice score evaluated on the reserved test set from the initial
image distribution; the 4 best-performing individual models, which
were tested in the ensemble-based approach, are in bold text.

| | Class | | | | |
|---|---|---|---|---|---|
| | | | Anterior | Posterior | Cervical |
| Model | Background | Bladder | Cervix | Cervix | Canal |
| SegResNet | 0.98 ∓ | 0.67 ∓ | 0.92 ∓ | 0.90 ∓ | 0.53 ∓ |
| | 0.02 | 0.23 | 0.02 | 0.09 | 0.26 |
| Attention | 0.97 ∓ | 0.68 ∓ | 0.91 ∓ | 0.89 ∓ | 0.49 ∓ |
| UNet | 0.02 | 0.21 | 0.04 | 0.10 | 0.23 |
| nn-UNet | 0.97 ∓ | 0.65 ∓ | 0.92 ∓ | 0.90 ∓ | 0.48 ∓ |
| | 0.03 | 0.23 | 0.03 | 0.09 | 0.24 |
| ResUNet 4 | 0.98 ∓ | 0.67 ∓ | 0.92 ∓ | 0.92 ∓ | 0.54 ∓ |
| | 0.02 | 0.24 | 0.02 | 0.05 | 0.24 |
| ResUNet 2 | 0.97 ∓ | 0.67 ∓ | 0.92 ∓ | 0.91 ∓ | 0.54 ∓ |
| | 0.03 | 0.23 | 0.02 | 0.06 | 0.23 |
| UNet | 0.96 ∓ | 0.28 ∓ | 0.88 ∓ | 0.87 ∓ | 0.52 ∓ |
| | 0.03 | 0.07 | 0.04 | 0.08 | 0.21 |
| UNETR | 0.95 ∓ | 0.53 ∓ | 0.78 ∓ | 0.75 ∓ | 0.41 ∓ |
| | 0.03 | 0.18 | 0.08 | 0.12 | 0.21 |

TABLE 5
Model training time. Training time required for each
model architecture, in an hour:minute:second format.

| Model | Training Time |
|---|---|
| SegResNet | 7:04:10 |
| Attention UNet | 8:01:11 |
| nn-UNet | 8:43:34 |
| ResUNet 4 | 9:00:19 |
| ResUNet 2 | 9:43:39 |
| UNet | 11:38:54 |
| UNETR | 29:58:25 |

TABLE 6
Similarity metrics for best performing SegResNet architecture;
comparisons on Dice metric (DM), Hausdorff distance (HD),
and Jaccard index (JI) are tabulated for the best performing
SegResNet architecture; Class-specific average metrics and
standard deviations are calculated across all images in the
reserved test set; *indicates average calculated over N =
25 samples because one sample did not have cervical canal class
and the associated Hausdorff distance was infinite.

| | Class | | | | |
|---|---|---|---|---|---|
| | | | Anterior | Posterior | Cervical |
| Metric | Background | Bladder | Cervix | Cervix | Canal |
| DM | 0.98 ∓ | 0.67 ∓ | 0.92 ∓ | 0.90 ∓ | 0.53 ∓ |
| | 0.02 | 0.23 | 0.02 | 0.09 | 0.26 |
| HD | 23.07 ∓ | 14.38 ∓ | 19.49 ∓ | 23.79 ∓ | *32.11 ∓ |
| | 16.58 | 7.94 | 18.26 | 23.11 | 18.59 |
| JI | 0.95 ∓ | 0.55 ∓ | 0.84 ∓ | 0.84 ∓ | 0.40 ∓ |
| | 0.04 | 0.23 | 0.04 | 0.12 | 0.24 |

TABLE 7
Similarity metrics for best performing nn-UNet architecture; Dice metric
(DM), Hausdorff distance (HD), and Jaccard index (JI) are tabulated
for the best performing nn-UNet architecture; Class-specific average
metrics and standard deviations are calculated across all images in
the test set; *indicates average calculated over N = 25 samples
because 1 prediction mask did not have any instance of cervical canal
class, resulting in an infinite associated Hausdorff distance.

| | Class | | | | |
|---|---|---|---|---|---|
| | | | Anterior | Posterior | Cervical |
| Metric | Background | Bladder | Cervix | Cervix | Canal |
| DM | 0.97 ∓ | 0.65 ∓ | 0.92 ∓ | 0.90 ∓ | 0.48 ∓ |
| | 0.03 | 0.23 | 0.03 | 0.09 | 0.24 |
| HD | 30.71 ∓ | 18.55 ∓ | 19.82 ∓ | 22.22 ∓ | *30.80 ∓ |
| | 16.98 | 23.09 | 15.04 | 17.55 | 16.33 |
| JI | 0.95 ∓ | 0.52 ∓ | 0.85 ∓ | 0.83 ∓ | 0.35 ∓ |
| | 0.05 | 0.24 | 0.04 | 0.11 | 0.21 |

TABLE 8
Similarity metrics for best performing Attention UNet architecture;
Dice metric (DM), Hausdorff distance (HD), and Jaccard index
(JI) are tabulated for the best performing Attention UNet architecture;
Class-specific average metrics and standard deviations are calculated
across all images in the reserved test set; *indicates average
calculated over N = 25 samples because 1 prediction mask
did not have any instance of cervical canal class, resulting
in an infinite associated Hausdorff distance.

| | Class | | | | |
|---|---|---|---|---|---|
| | | | Anterior | Posterior | Cervical |
| Metric | Background | Bladder | Cervix | Cervix | Canal |
| DM | 0.97 ∓ | 0.68 ∓ | 0.91 ∓ | 0.89 ∓ | 0.49 ∓ |
| | 0.02 | 0.21 | 0.04 | 0.10 | 0.23 |
| HD | 24.79 ∓ | 18.90 ∓ | 23.60 ∓ | 26.39 ∓ | *32.03 ∓ |
| | 15.19 | 23.43 | 24.90 | 24.13 | 18.28 |
| JI | 0.95 ∓ | 0.55 ∓ | 0.84 ∓ | 0.81 ∓ | 0.35 ∓ |
| | 0.04 | 0.22 | 0.07 | 0.13 | 0.21 |

TABLE 9
Similarity metrics for best performing Residual UNet architecture;
Dice metric (DM), Hausdorff distance (HD), and Jaccard index (JI)
are tabulated for the best performing Residual UNet architecture with
2 residual units; Class-specific average metrics and standard deviations
are calculated across all images in the reserved test set.

| | Class | | | | |
|---|---|---|---|---|---|
| | | | Anterior | Posterior | |
| Metric | Background | Bladder | Cervix | Cervix | Cervical Canal |
| DM | 0.97 ∓ | 0.67 ∓ | 0.92 ∓ | 0.91 ∓ | 0.54 ∓ |
| | 0.03 | 0.23 | 0.02 | 0.06 | 0.23 |
| HD | 24.33 ∓ | 17.13 ∓ | 18.40 ∓ | 23.15 ∓ | 30.93 ∓ |
| | 14.97 | 24.00 | 14.96 | 21.16 | 18.75 |
| JI | 0.95 ∓ | 0.54 ∓ | 0.85 ∓ | 0.84 ∓ | 0.40 ∓ |
| | 0.05 | 0.24 | 0.04 | 0.09 | 0.21 |

Statistical Tests: Normal curves were fit to the CL distributions and overlaid on the same graph (FIG. 15). Most images have a positive percent error (FIG. 16), indicating the algorithm-reported value is larger than the sonographer-reported value; this aligns with the slightly rightward-skewed CL normal curve and is expected because the expert measurements were taken as a series of line segments, whereas the algorithm was permitted to follow inherently longer curvilinear traces.

Given the small size of the reserved test dataset, the performance metrics are not assumed to follow a normal distribution. Therefore, non-parametric statistical tests were used to test the null hypothesis that the performance metrics for each model were drawn from the same underlying distribution. One-way paired Friedman test was used to detect differences between the performance across all models. The Friedman test indicated a difference between mean performance metrics across all model types. A paired multiple comparison Wilcoxon Signed-Rank test with Bonferroni corrections was used to compare the performance of each model in terms of Dice metric, Hausdorff distance, and Jaccard index. All comparisons were made with consistent results across the Dice metric, Hausdorff distance, and Jaccard index. Hausdorff distance indicated a difference between basic UNet and transformer UNet (p<0.01), whereas Dice metric and Jaccard index indicate no difference between the performance of basic UNet and transformer UNet.

To confirm that these cervical length values were drawn from the same distribution, a Wilcoxon signed rank test was performed with the null hypothesis that there is no difference between the average sonographer reported and corresponding algorithmic reported cervical length value. The test failed to reject the null hypothesis, indicating that the algorithm and the sonographer measurements are drawn from the same cervical length distribution.

FIG. 15 shows the normal distribution of CL measurements. Normal curves fit to TVU CL distribution, plotted for each expert and algorithm measurement, demonstrate high agreement among experts and algorithm output, with the algorithm distribution being slightly wider and skewed rightward.

FIG. 16 shows the percent error in the CL algorithm. Percent error (PE) is plotted for each patient, where algorithm-derived CL is compared against sonographer-measured CL. A negative PE indicates algorithmic CL is smaller than sonographer CL measurements, whereas a positive PE indicates algorithmic CL is larger than sonographic CL.

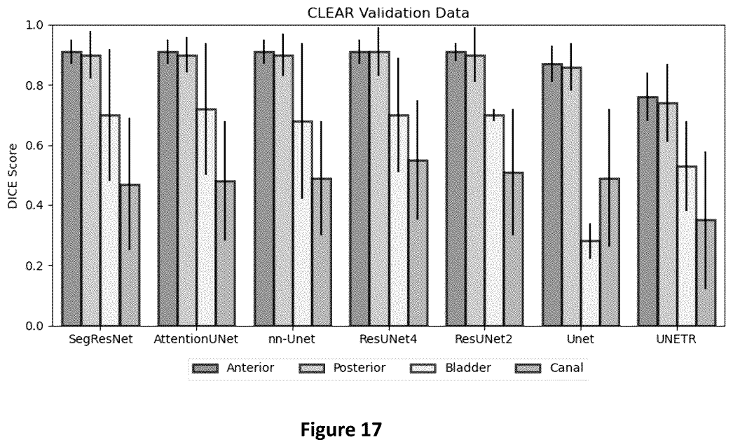

FIG. 17 shows the ensemble model performance on CLEAR validation data. All models are compared using class-specific Dice scores, averaged across all images in the validation set from the original CLEAR dataset. Error bars indicate 1 standard deviation from the mean across images in the test set. Models are ordered from left to right in terms of descending time required for model training.

FIG. 18 shows the individual model comparisons on varied cervical geometry. Ground truth and prediction labels in the validation set demonstrate good model performance for variations in cervical geometry, including a) long curved cervix, b) short squat cervix, and c) median width/length cervix. Each model is represented with row-wise predictions on these 3 cervix phenotypes. Prediction rows are in descending order with respect to the time required for training.

FIG. 19 shows the attention to UNet model segmentation on CLEAR validation data. Ground truth and prediction labels in the validation set demonstrate good Attention UNet model performance for variations in cervical geometry, including a) long curved cervix, b) short squat cervix, and c) median width/length cervix. Image-specific dice scores are reported according to class color. d) Training and validation mean dice score (top) and loss (bottom) are plotted against the number of epochs to visualize model training.

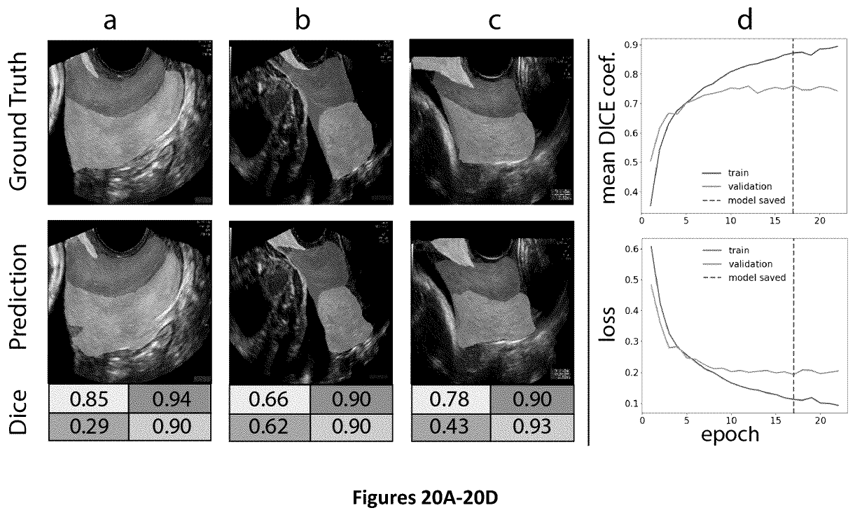

FIG. 20 shows the nn-UNet model segmentation on CLEAR validation data. Ground truth and prediction labels in the validation set demonstrate good nn-UNet model performance for variations in cervical geometry, including a) long curved cervix, b) short squat cervix, and c) median width/length cervix. Image-specific dice scores are reported according to class color. d) Training and validation mean dice score (top) and loss (bottom) are plotted against the number of epochs to visualize model training.

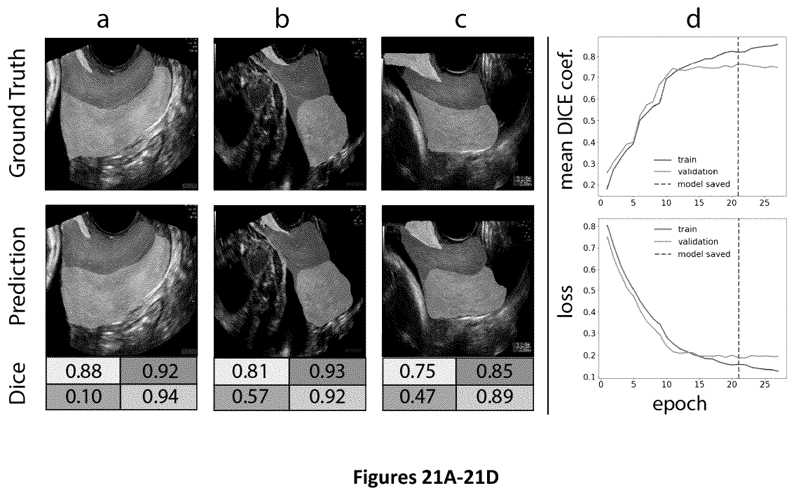

FIG. 21 shows the SegResNet model segmentation on CLEAR validation data. Ground truth and prediction labels in the validation set demonstrate good SegResNet model performance for variations in cervical geometry, including a) long curved cervix, b) short squat cervix, and c) median width/length cervix. Image-specific dice scores are reported according to class color. d) Training and validation mean dice score (top) and loss (bottom) are plotted against the number of epochs to visualize model training.

FIG. 22 shows the basic UNet model segmentation on CLEAR validation data. Ground truth and prediction labels in the validation set demonstrate good Residual UNet (0 residual units) model performance for variations in cervical geometry, including a) long curved cervix, b) short squat cervix, and c) median width/length cervix. Image-specific dice scores are reported according to class color. d) Training and validation mean dice score (top) and loss (bottom) are plotted against the number of epochs to visualize model training.

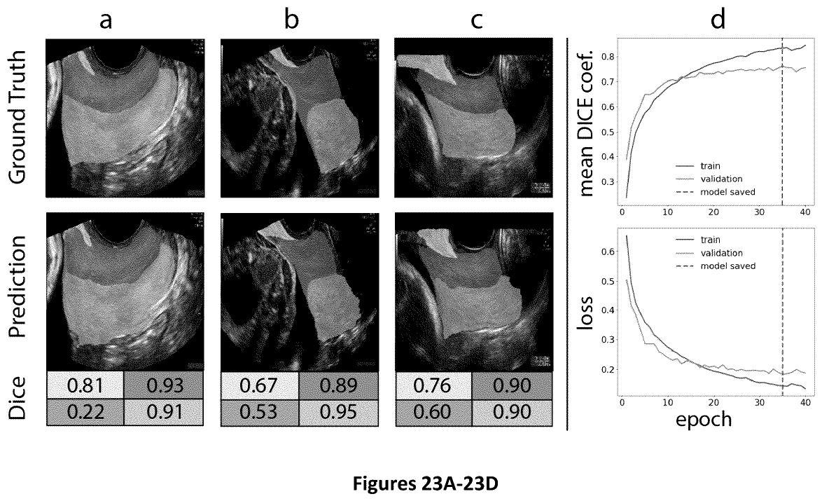

FIG. 23 shows the 2-unit Residual UNet model segmentation on CLEAR validation data. Ground truth and prediction labels in the validation set demonstrate good Residual UNet (2 residual units) model performance for variations in cervical geometry, including a) long curved cervix, b) short squat cervix, and c) median width/length cervix. Image-specific dice scores are reported according to class color. d) Training and validation mean dice score (top) and loss (bottom) are plotted against the number of epochs to visualize model training.

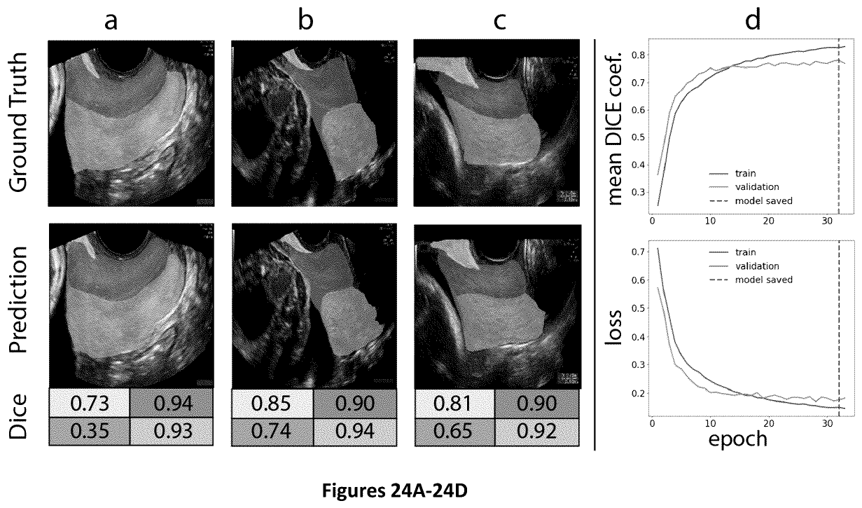

FIG. 24 shows the 4-unit Residual UNet model segmentation on CLEAR validation data. Ground truth and prediction labels in the validation set demonstrate good Residual UNet (4 residual units) model performance for variations in cervical geometry, including: a) long curved cervix, b) short squat cervix, and c) median width/length cervix. Image-specific dice scores are reported according to class color. d) Training and validation mean dice score (top) and loss (bottom) are plotted against the number of epochs to visualize model training.

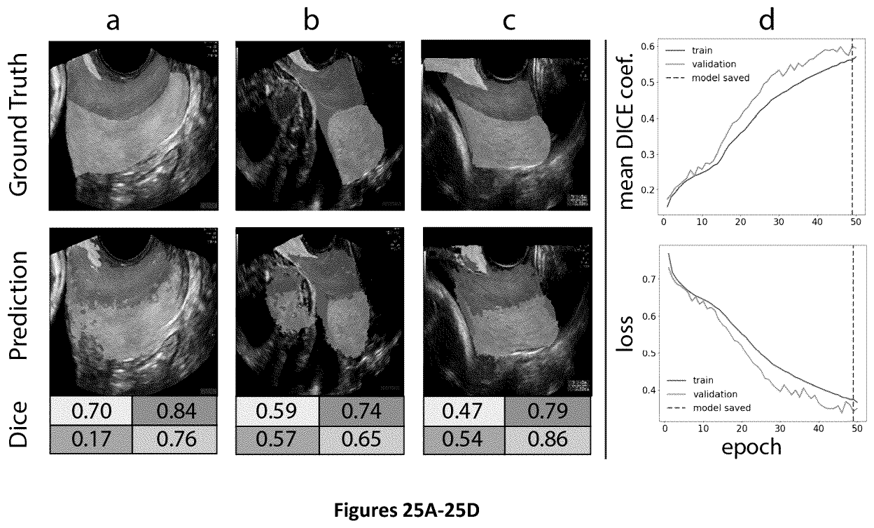

FIG. 25 shows the transformer UNet model segmentation on CLEAR validation data. Ground truth and prediction labels in the validation set demonstrate mediocre transformer UNet model performance for variations in cervical geometry, including a) long curved cervix, b) short squat cervix, and c) median width/length cervix. Image-specific dice scores are reported according to class color. d) Training and validation mean dice score (top) and loss (bottom) are plotted against the number of epochs to visualize model training.

FIG. 26 shows the out-of-distribution test set images excluded from the analysis. Ultrasound images (row 1) and corresponding segmentation masks (row 2) shown here were excluded from analysis due to shadowing and/or artifact.

The disclosed AI-enabled segmentation of the cervix and related anatomy facilitated automated CL measurements that were performed as well as experts. The disclosed AI tools were utilized in a protocol to measure TVU CL and evaluate adjacent anatomy in pregnant persons.

TVU CL is considered the only imaging biomarker of sPTB risk. The disclosed AI tools for CL measurement can benefit settings lacking highly trained operators. The disclosed subject matter catalyzes the development of AI-enabled methods to interpret multiple features of maternal anatomy, potentially pushing our capabilities beyond simple CL measurement toward biomechanically-informed decisions about sPTB risk based on an individual's unique geometry.

The cervix is a complex, 3D structure that, in normal pregnancy, initially maintains the growing fetus in utero and subsequently remodels to release it at term. This process, though certainly driven by molecular processes, is fundamentally biomechanical. Premature cervical shortening, a common feature of sPTB, can be thought of as a structural “failure” of the tissue. Biomechanical models explain 3D tissue behaviors by determining how overall shape, volume, intrinsic material properties, and alignment between the cervix/uterus against the load of the growing fetus affect mechanical performance.

Automation of CL measurement is an important advance and can lead to improved predictive capability, but its low PPV indicates that this 2D measurement cannot sufficiently capture the 3D biomechanics of cervical preparation for delivery. The disclosed image segmentation tool, however, allows the extraction of multiple geometric features that aid in the 3D reconstruction of the cervix and LUS. Future integration of features, such as cervical diameter, cervical curvature, AUCA, LUS thickness, closed cervical area, and others, has broad applications for understanding precise, patient-specific maternal geometry and implications for the timing of delivery.

The diverse, multi-institution training data with known quality measures (CLEAR scores) and multiple expert labels were used to develop a segmentation model. In addition to the accurate performance on the original datasets, similar performance on a separate clinical dataset, drawn from a different distribution, reinforces trust in model generalizability. Accordingly, the model can be used for new multi-site, diverse demographic data.

The disclosed subject matter provides an automated, multi-class segmentation network to label cervical tissue in its entirety on TVU images and automate cervical length measurement. Compared to other techniques that segmented only 1 class approximating the cervix, the disclosed multi-class ensemble model expands and achieves a similar Dice score of 0.93 and 0.92 for both anterior and posterior cervix classes on in-distribution data. The model was deployed on an out-of-distribution dataset for the first time and maintained high model performance with a Dice score of 0.80 and 0.84 for the anterior and posterior cervix classes, respectively.

The disclosed subject matter can be used to predict sPTB and compared against clinical outcomes. The disclosed subject matter can allow an engineering-based method to predict sPTB via interrogating biomechanical mechanisms of birth, ultimately translating into a practical clinical workflow for modern obstetric care.

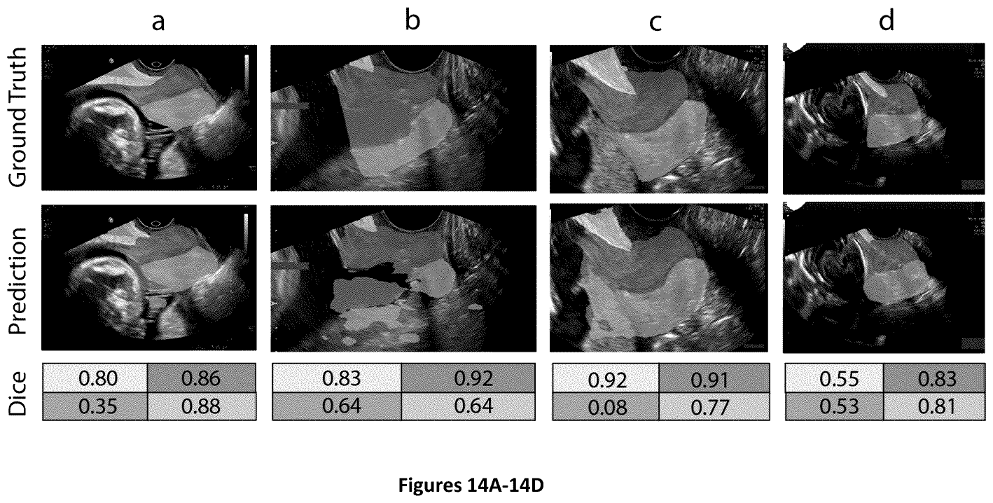

The bladder, while holding little meaning as a stand-alone feature, can also act as a helpful landmark to aid a cervical feature extraction model. Although bladder predictions were less reliable than cervix predictions, the inclusion of the bladder class in this model can improve the overall performance by providing a reliable, highly echogenic landmark with an anatomically prescribed location near the anterior/superior boundary of the cervix. Similarly, the cervical canal class can also be used to examine the shape and size of a funnel or cervical mucus plug if present in the TVU image. In both the original and the out-of-distribution datasets, many segmentation masks underpredict the inferior-most aspect of the bladder flap. While the predicted bladder pixels can be in the correct location, this systematic underprediction of the bladder flap effectively lowers the Dice score for the bladder class. In select images, such as the atypical cervix with a large cervical funnel shown in FIG. 14, there can be disjointed regions and, therefore, multiple instances of the same class. FIG. 14 shows the model performance challenges for validation images where certain artifacts limit reliability: (14A) The presence of a full bladder, (14B) funneling, (14C) a low-lying placenta, and (14D) an extremely zoomed-out field of view hinder model performance.

Anatomically, this is an impossibility and therefore, a post-processing procedure is warranted to correct for small disconnected regions. With the disclosed subject matter, certain post-processing procedures were performed to remove these disjointed regions or “islands” from the segmentation masks before applying the cervical length algorithms.

### Example 2: Cervical Feature Extraction from Segmented Transvaginal Ultrasound Images

The disclosed subject matter provides a cervical feature extraction tool to assist in automating the process of measuring cervical features that can play a role in the prediction of preterm birth. The feature extraction tool uses standard-of-care transvaginal ultrasounds that have been segmented into four classes (cervical canal, anterior cervix, posterior cervix, and bladder) by three experts. Then, using image processing techniques, measurements were found for cervical length, lower uterine segment thickness, anterior uterocervical angle, and anterior and posterior cervical diameter at multiple locations. Results were validated for cervical length measurements.

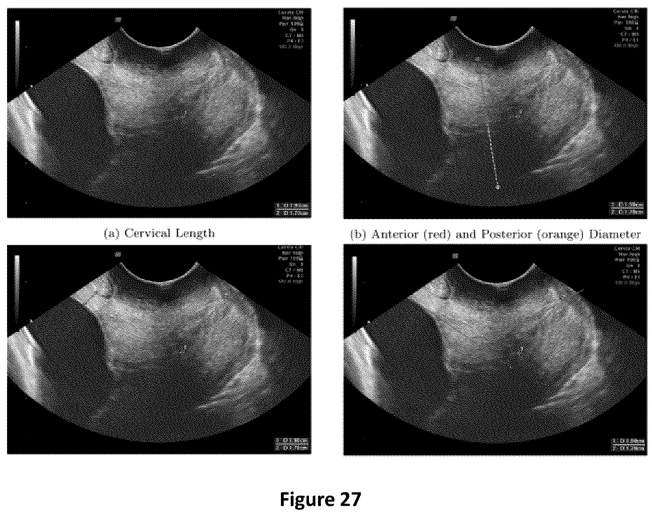

Cervical length is defined as the distance from the internal os to the external os following the curvature of the cervix. Anterior and posterior cervical diameter refers to the width of the cervix in the anterior and posterior portions of the sagittal cross-sectional view of the cervix. Lower uterine segment thickness is the thickness of the lower aspect of the anterior uterus. Anterior uterocervical angle is the angle created by the anterior cervix and lower uterine segment (FIG. 27). In some cases, there are variations in how these measurements are taken. For example, cervical length is often measured clinically as one straight line segment connecting the internal and external os despite the fact that oftentimes the cervical canal is curved. Another example of this is the ambiguity surrounding the correct way to measure the AUCA.

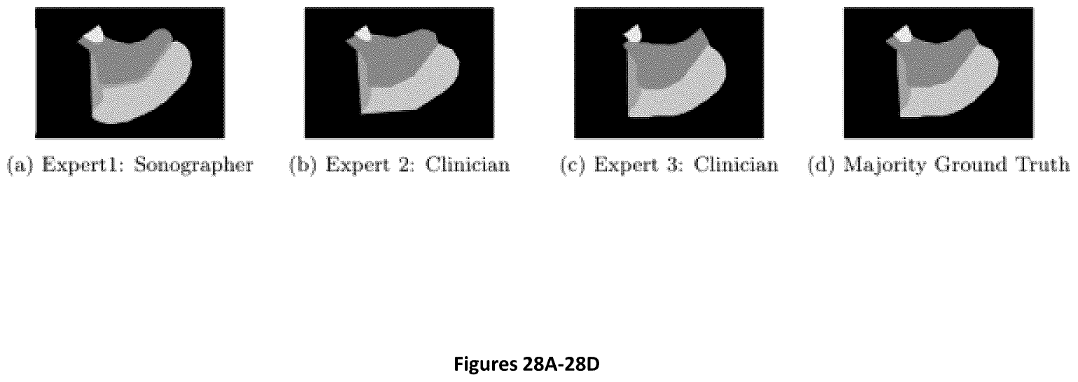

Data Set: To prepare the ultrasounds for the automated cervical feature extraction process that is under development, the images were segmented into four classes: anterior cervix and lower uterine segment, posterior cervix, bladder, and cervical canal. The images used to develop the algorithms for feature extraction were manually segmented by two clinicians and a sonographer, resulting in three expert opinions on the segmentation of the image. The data set included 250 CLEAR-certified TVUS images taken between 16 and 32 weeks of gestation. During expert labeling, four images were excluded due to poor quality, leaving 246 labeled ultrasounds. A fourth opinion was created by combining the segmentation of each of the experts by using a majority voting approach. To illustrate, if at least two out of three experts labeled a pixel as part of the cervical canal, that pixel would be labeled as a cervical canal in the majority of ground truth segmentation (FIG. 28). After this process, the 250 original ultrasounds result in 984 segmentation masks to use to develop a cervical feature extraction algorithm.

Pre-processing: Each feature was measured with a different technique, but pre-processing was applied to each image before any measurements were taken. First, the image was converted to a grayscale. Then, the classes that are present in a specific image are identified; in some cases, not every class appears in every image (in a few cases, the bladder or the cervical canal class was not labeled). Class detection was performed based on pixel color values in the segmentation mask.

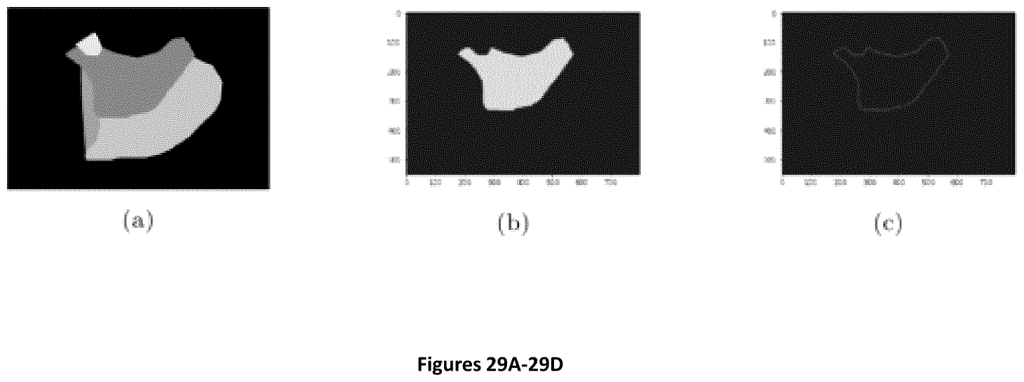

Next, a single-class mask (example shown in FIG. 29B) was created for each identified class in an image. Finally, the border was extracted from each single-class mask (as shown in FIG. 29C).

Cervical Length: The first procedure in measuring the cervical length of an image is to locate the internal and external os, which are the openings at the inferior and superior end of the cervical canal. The process of locating the internal os is not straightforward because many cervices have a funnel-like structure at the opening; therefore, the end of the funnel must be located to determine where the cervical canal starts. The method proposed for locating this point depends on how the ultrasound was labeled by the experts.

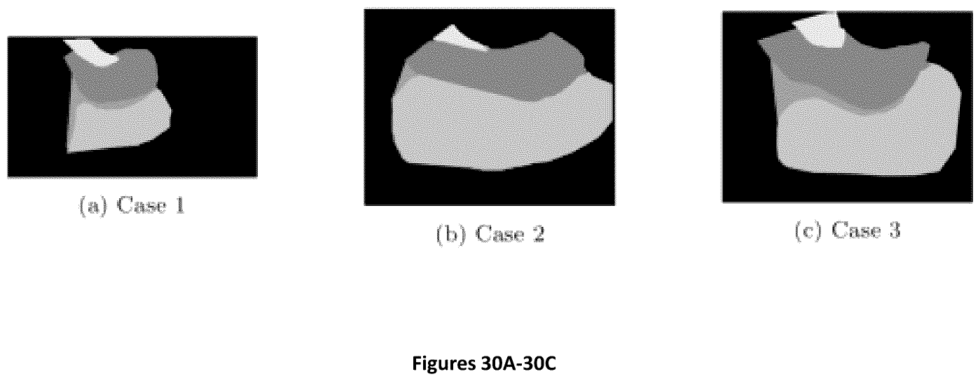

When provided with the same segmentation criteria, one of the experts frequently labeled the cervical canal all the way from internal to external os (FIG. 28A), while the other two experts tended to only label the funnel/cervical canal opening near the internal os. In some cases, the cervical canal class label was also labeled as a mixture of these two, with the class extending partway between the internal and external os. Because of these different labeling patterns, first, the segmentation was classified as either case 1, case 2, or case 3, respectively (FIG. 30).

For each case, the column-wise length of the cervical canal class along the horizontal length of the image was considered. In Case 1, the cervical canal class presents with a funnel towards the left of the image which rapidly narrows before leveling off below a certain threshold. In the columns corresponding to the funnel, the top and bottom pixels in each column are rapidly getting closer as the funnel narrows, but once the cervical canal starts, the top and bottom pixels in each column stay a relatively similar distance apart. The internal os was chosen as the point where this rapid narrowing behavior of the funneling stops and the cervical canal begins. This was done by measuring the distance between the top and bottom pixels of the cervical canal class in each column and comparing this distance to the proceeding (right-hand) column. If the distance between the top and bottom pixels of the cervical canal class was the same for three columns in a row, the internal os was labeled at the vertical midpoint between the two pixels in the first column. It is assumed that if the distance between pixels is not changing from column to column, this implies that the structure has flattened out and is no longer funneling. Identifying the internal os in Case 2 images is more straightforward because the expert stopped labeling the cervical canal class precisely where the funneling behavior ends. In this case, the internal os is located at the first point where the anterior cervix class touches the posterior cervix class. Otherwise, if the cervical canal class is not present, the leftmost point where the anterior/posterior cervix classes touch is taken as the internal os. For complex presentations in Case 3 images, the method from Case 1 is used to find where the funneling behavior stops.

Next, the external os was identified. In Case 1 masks, the external os was located by identifying the column that contains the highest cervical canal point in the rightmost 25% of the cervical canal. In Case 2 or 3 masks, the external os was located at the last point that the anterior and posterior cervical classes touch.

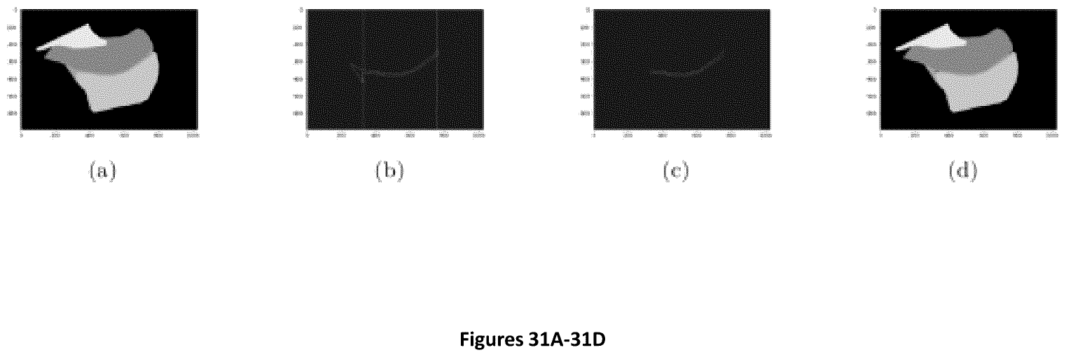

The cervical length can be measured using the locations of the internal and external os as well as the border images generated (FIG. 29C). If the image has been classified as Case 1, all the pixels (in the border image) to the left of the internal os and to the right of the external os are removed, leaving only the two lines that make up the top and bottom of the cervical canal (FIG. 31C). The length of these two lines is then averaged, and the resulting value is reported as the cervical length. If the image has been classified as Case 2, the cervical length (in pixels) is the number of pixels where the anterior and posterior cervix classes are touching (FIG. 29). If the image has been classified as Case 3, the cervical length is the number of pixels where the anterior and posterior cervix classes touch in addition to the distance between the internal os and the leftmost point where the anterior and posterior cervix touch (FIG. 30C). This is necessary because the internal os in Case 3 masks is not strictly located at the point leftmost point where the anterior and posterior cervix touch, as it is in Case 2.

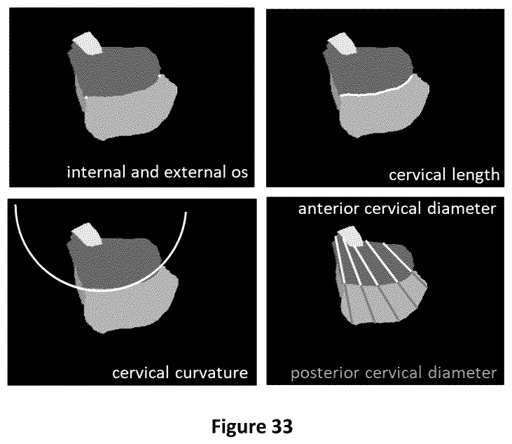

Anterior and Posterior Cervical Diameter: The anterior and posterior cervical diameter can be measured anywhere along the cervical length, so the first procedure to taking this measurement is to decide where along the cervical length. Once that has been specified as a percentage of the cervical length (meaning that 50% would correspond with a measurement that is taken halfway along the cervical length), that point is located in the image. If it is a case 1 image where the anterior and posterior cervix do not touch along the cervical length, the anterior boundary of the green cervical canal class was used to determine the measurement locations along the cervical length. In case 2 images where the cervical length was defined along the intersection of anterior/posterior classes, cervical diameter measurements were taken at points along (25%, 50%, etc.) cervical length following this intersection. In case 3 images, measurement was taken along the combined straight line from internal os to where the anterior/posterior cervix class touches and the curved line following where the anterior/posterior cervix touches. Next, the roughly five (depending on the length of the cervix) pixels to the left and right are collected and used to fit a line that is tangent to the cervix. Once that line has been found, a perpendicular line that originates at the initial point at the specified percentage is drawn until it intersects the other side of the cervix border. The distance between the point specified along the cervical length and the intersection point is calculated and returned as the diameter (FIG. 33).

Lower Uterine Segment Thickness: It is difficult to consistently measure lower uterine segment thickness across different ultrasounds due to inconsistencies and irregularities in shape. As a result, the visual measurements of LUS produced by this algorithm can appear unusual in some outlier cases. The existence of these unusual shapes is known and preferred to inconsistent methods to measure LUS thickness. The proposed methodology to consistently measure LUS thickness first finds a perpendicular slope to the LUS. Then, the midpoint of the posterior boundary of the bladder is located. Finally, the identified perpendicular slope is used to draw a line connecting the bladder midpoint to the intersection point on the posterior boundary/line of the LUS. The distance of this line, connecting the midpoint of the bladder and the spot where the perpendicular line intersects the LUS, is taken as the LUS thickness.

This method was implemented by first finding the superior-most labeled point on the posterior wall of the anterior lower uterine segment and 5-10 points neighboring it along the posterior wall. These points were then used with a linear regression algorithm to fit a line that is parallel to the posterior wall of the lower uterine segment. From that line, the perpendicular slope was found, and a linear equation was created using that slope and an intercept of the midpoint of the posterior bladder wall. This line does not always intersect the portion of the LUS that is labeled, so instead of using that intersection as the second point for the distance formula, the point used, in addition to the point on the bladder, is the point at which the perpendicular line intersects the line that is parallel to the LUS. Once these two points have been found, the lower uterine segment thickness is calculated.

Anterior Uterocervical Angle: This feature is unique in the fact that across the literature that defines this angle, there are many ambiguities regarding how the angle can be properly drawn on an image for measurement. Therefore, before discussing the approach used for extracting this feature, it is important to explore the various ways it could be measured. Existing literature agrees that the first line used to form the AUCA can be drawn by connecting the internal os to the external os, but the location of the second line is not clearly defined. In particular, there are two definitions of the AUCA that are referenced more often than others. One frequently cited definition specifies: (1) The second line can be traced up the anterior uterine segment to delineate the LUS; (2) In the case of funneling, the second line can originate at the internal os and be extended to the LUS; (3) If the LUS is irregular, the line can be drawn from the internal os to a point located centrally along the segment; (4) The second line can be drawn parallel to the lower aspect of the LUS, passing through the internal os.

While initially these definitions seem reasonable, and they work for cervices without irregularities in their structure, complications in finding a consistent angle arise when structures like funneling are present. While the first definition does define what to do in the case of funneling, it is not consistent with the naming convention of anterior uterocervical angle, which suggests the angle will be defined between the anterior uterus and the cervix. This inconsistency in name and definition can lead to confusion. On the other hand, the second definition offers no solution if funneling is present. Because of this, papers citing these definitions often provide example figures that are either inconsistent with their referenced definition, inconsistent with other papers citing the same method, or provide a figure that shows the angle drawn on an unambiguous cervical structure, which perpetuates uncertainties when more complex presentations arise.

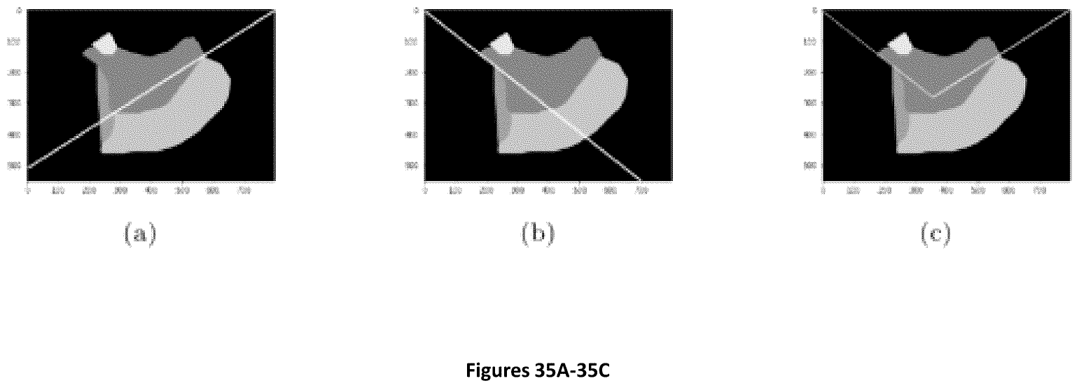

For the proposed feature extraction tool, AUCA was defined in a way that is consistent with its name, implying it to be the angle between the anterior uterus and the cervix. Because of the nature of how the ultrasounds were labeled by the experts (who were asked to label the pink anterior cervix and LUS class in the superior direction enough that a measurement could be collected of the anterior LUS thickness based on the provided label), the leftmost points on the posterior wall of the anterior cervix class are points on the LUS. Therefore, a line fit to these points represents the orientation of the uterus; this is the same line that was used in the measure of LUS thickness. Similar to the methods proposed in existing literature, the orientation of the cervix is found by drawing a line through the internal and external os. The AUCA is then measured as the angle between these two lines at the point where these two lines intersect, as shown in FIG. 35C.

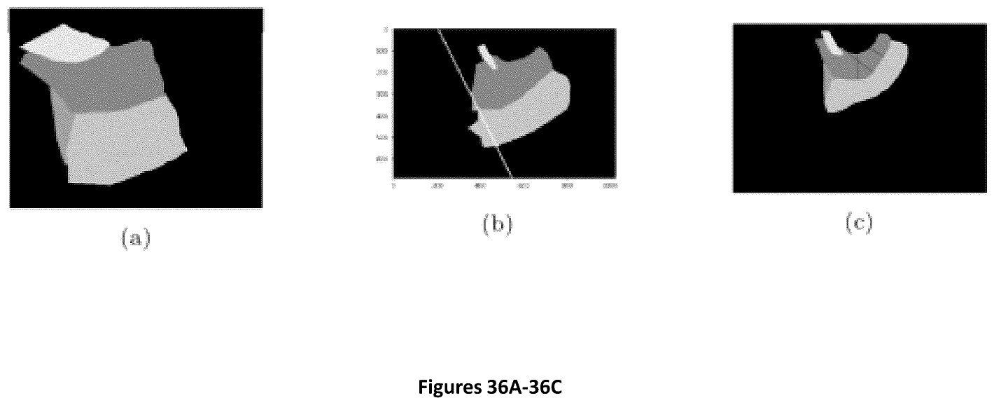

When measuring anterior and posterior diameter, if the cervical canal is particularly curved, the lines drawn perpendicularly to it often either intersect with each other or intersect the opposite side (superior vs. inferior) of the cervix in a location that is inconsistent with the desired measurement (FIG. 36).

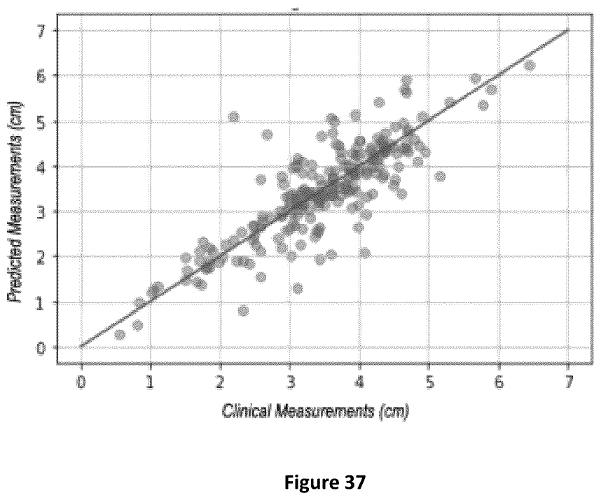

In FIG. 37, the line in the scatter plot that compares predicted values and clinical measurements is a guide to show where the points lie if predicted values are the same as clinical measurements, and the dots represent the actual predicted values and clinical measurements for ground truths images.

Validation: Since cervical length is the only clinically measured feature, validating the results of all the feature extraction methods is not yet possible, but cervical length measurements were taken for most of the ultrasounds used to create this tool. We compared the predicted cervical length to the sonographic cervical length reported on the underlying ultrasound image itself. To generate these comparisons, the cervical length feature extraction method was used on all ground truth labels. Because of the limitations addressed in FIG. 36, 7 images were removed due to errors in the code, and 12 images did not have clinical cervical measurements. Using the clinically measured cervical length as the expected cervical length, the cervical length feature extraction method had a 15 percent error. A paired T-test was used to find the difference between the cervical length distributions was not statistically significant (pᵢ0.05). A normal cervical length distribution was assumed and can be validated by using this statistical test.

The disclosed subject matter provides a cervical feature extraction tool to measure cervical features such as cervical length, lower uterine segment thickness, anterior and posterior cervical diameter, and anterior uterocervical angle. Measuring these features by hand is time-consuming, not scalable, and potentially inconsistent, which results in a lack of research surrounding the significance of these measurements in predicting preterm birth. While there was not a robust way to validate results with this specific data set, the tool will hopefully be re-evaluated in the future with another data set, including clinical measurements for features besides cervical length. Other future work includes exploring more features such as closed cervical area and cervical canal curvature. Furthermore, this tool can be used in combination with automatic deep learning-based segmentation methods to automate the entire process of extracting these measurements. As a result, these features of cervical geometry and their significance in predicting preterm birth could be studied in depth, and eventually, this automated tool could even be used clinically because of its adaptability to fit within the stringent time schedule of clinical evaluation.

### Example 3: A Fast, Reliable, and Quantitative Assessment of Maternal Anatomy for Measuring Cervical Structural Health and the Amount of Load on the Cervix

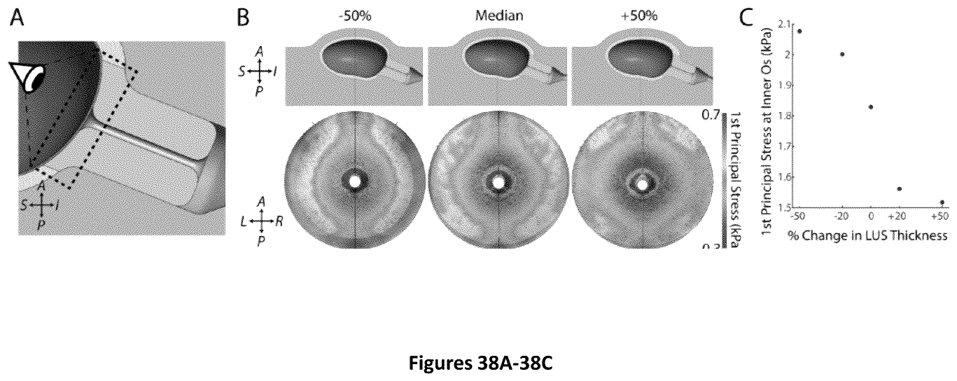

The cervix is a mechanical barrier for the fetus, where it serves as structural support and protects from ascending infection. Premature remodeling, shortening, and dilation of the cervix can be the pathway for etiologies of spontaneous PTB. Three-dimensional biomechanical models (FIGS. 38A-38C) revealed that cervical length only partially determines the overall biomechanical performance of the cervix. The disclosed subject matter provides a model built using finite element analysis (FEA), which can be a computational engineering model that calculates the amount of stress and stretch within a structure given the magnitude and direction of mechanical loading and the intrinsic elasticity (i.e., material properties) of the structure. When considering the cervix as a structure, an FEA simulation can quantify and map the amount of cervical stress and stretch based on the pressure exerted by the fetal membranes, the pull of the uterine wall, the intrinsic elasticity of the cervical tissue, and the adhesive quality and stiffness of the fetal membrane.

Collectively, the disclosed FEA simulations can provide mechanistic insight into which biophysical factors cause the cervix to stretch, shorten, funnel and dilate. In a large-scale sensitivity analysis of biomechanical factors, cervical size, cervical shape and lower uterine segment thickness influence the distribution and magnitude of tissue stretch. Calculations of biomechanical stress within the cervix (FIG. 38) show that thinning of the lower uterine segment increases the tensile pull of the uterine wall, promoting large mechanical stresses that drive cervical stretching and membrane funneling. As for cervical structural integrity, cervical elasticity, size, and shape influence how the cervix counteracts mechanical loads. Based on physics, cervical structural integrity increases with tissue volume. Cervical tissue volume can be estimated from a transvaginal ultrasound by measuring the closed cervical area and cervical diameter. Additionally, lower uterine segment thickness can be measured from a transvaginal ultrasound.

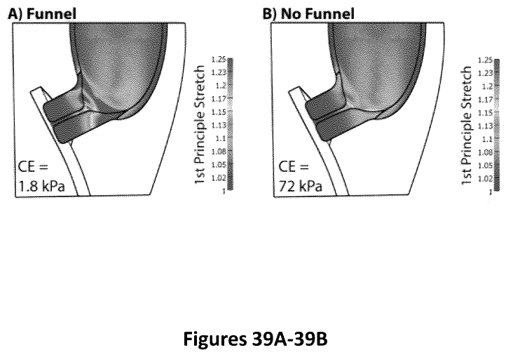

In addition to cervical shape and size, intrinsic cervical elasticity can also contribute to cervical structural integrity. The disclosed FEA model shows that a decrease in cervical elasticity produces large cervical tissue stretches and membrane funneling (FIG. 39). The disclosed subject matter provides tools to measure cervical elasticity (i.e., stiffness). The tools can include cervical aspiration and quantitative ultrasound. The aspiration tool can measure cervical tissue stiffness by applying a small negative pressure on the external cervical os and pulling the tissue until it touches a 4 mm stop. The aspiration closure pressure can be recorded, where a higher value of closure pressure corresponds to a stiffer tissue. In a prospective cohort assessment of pregnant patients with an incidental short cervix, patients who delivered preterm (n=6) had lower average cervical closure pressure (47.4 vs. 82.9 mbar) compared to patients who delivered at term (n=30). Shear wave elastography can be used to measure cervical softening throughout pregnancy with ultrasound transducer and system software to estimate shear wave speed within the cervix. The shear wave speed can be proportional to the mechanical shear modulus (i.e., cervical elasticity). Using both of these tools, cervical stiffness in real-time in pregnancy can be monitored, but interpreting the results can be complex because both measurements can be influenced by cervical shape and size. Hence, a detailed map of cervical shape and size that the disclosed subject matter provides can aid in the accuracy and reliability of cervical stiffness measurements and increase the adoption of this technology.

All patents, patent applications, publications, product descriptions, and protocols cited in this specification are hereby incorporated by reference in their entirety. In case of a conflict in terminology, the present disclosure controls.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain, using no more than routine experimentation, many equivalents to the specific embodiments described herein. Such equivalents are intended to be encompassed by the following claims.

What is claimed is:

1. A method for predicting a spontaneous preterm birth based on transvaginal ultrasound images of a subject, comprising:
providing a preterm birth prediction model based on a plurality of biomechanical records including cervical features;
obtaining one or more transvaginal ultrasound images of the subject, each including cervical features;
determining measurements of a plurality of cervical structure features from the one or more ultrasound images;
assessing, using the preterm birth prediction model, cervical health of the subject based on the measurements of the plurality of cervical structure features; and
calculating, using the preterm birth prediction model, the spontaneous preterm birth risk based on the assessed cervical health.

2. The method of claim 1, wherein the preterm birth prediction model comprises a deep learning algorithm to identify cervical shape, size and load information.

3. The method of claim 2, wherein the deep-learning algorithm is trained on the plurality of biomechanical records to extract shape features and comparing extracted shape features against expert-reported features.

4. The method of claim 1, wherein the one or more transvaginal ultrasound images include unique pixel color values indicating segmentation of geometric features.

5. The method of claim 1, wherein the determining measurements of a plurality of cervical structure features comprises determining measurements selected from the group consisting of a cervical length, a lower uterine segment thickness, a cervical diameter, an anterior cervical diameter, a posterior cervical diameter and an anterior uterocervical angle.

6. The method of claim 5, wherein the determining measurements of the cervical length further comprises determining a distance between an internal and an external end of a cervical canal of the subject.

7. The method of claim 6, wherein the determining measurements of the anterior cervical diameter further comprises a measurement along the cervical length.

8. The method of claim 7, wherein the determining measurements of the posterior cervical diameter further comprises a measurement along the cervical length.

9. The method of claim 8, wherein the determining measurements the cervical diameter further comprises a measurement at an intersection of the anterior cervical diameter and the posterior cervical diameter.

10. The method of claim 5, wherein the determining measurements further comprises determining a perpendicular slope to the lower uterine segment and a midpoint of a posterior boundary of the subject's bladder.

11. The method of claim 10, wherein the determining measurements of the lower uterine segment thickness further comprises a measurement between the midpoint of the bladder and an intersection between a perpendicular line to the lower uterine segment.

12. The method of claim 5, wherein the determining measurements of the anterior uterocervical angle further comprises a measurement of an angle between the subject's anterior uterus and the cervix.

13. The method of claim 1, further comprising measuring cervical stiffness of the subject using a thin aspiration tube applied during a prenatal pelvic exam, and wherein the assessing cervical health of the subject is based on the measurements of the plurality of cervical structure features and the measured cervical stiffness.

14. The method of claim 1, wherein calculating the spontaneous preterm birth risk comprises generating a risk score.
